SEQUENCE LISTING (32 sequences)

SEQ ID NO: 1 - protein, 25 aa, Mus musculus
ILPQAPSGPSYATYLQPAQAQMLTP

SEQ ID NO: 2 - protein, 24 aa, Mus musculus
KPLRRNNSYTSYIMAICGMPLDSF

SEQ ID NO: 3 - protein, 25 aa, Mus musculus
PGPQNFPPQNMFEFPPHLSPPLLPP

SEQ ID NO: 4 - protein, 25 aa, Mus musculus
VIQTSKYYMRDVIAIESAWLLELAP

SEQ ID NO: 5 - protein, 25 aa, Mus musculus
SWIHCWKYLSVQSSQLFRGSSLLFR

SEQ ID NO: 6 - protein, 25 aa, Mus musculus
LLPFYPPDEALEIGLELNSSALPPT

SEQ ID NO: 7 - protein, 25 aa, Mus musculus
HIHRAGGLFVADAIQVGFGRIGKHF

SEQ ID NO: 8 - protein, 25 aa, Mus musculus
LRTAAYVNAIEKIFKVYNEAGVTFT

SEQ ID NO: 9 - protein, 17 aa, Mus musculus
ISQAVHAAHAEINEAGR

SEQ ID NO: 10 - protein, 18 aa, Mus musculus
DEVSGLEQLESIINFEKL

SEQ ID NO: 11 - protein, 20 aa, Artificial Sequence
  IL12 sp
MYSMQLASCVTLTLVLLVNS

SEQ ID NO: 12 - protein, 265 aa, Artificial Sequence
  CEA
MGSPSACPYRVCIPWQGLLLTASLLTFWNLPNSAQTNIDVVPFNVAEGKEVLLVVHNESQNLYGYNWYKGERVHANYRIIGYVKNISQENAPGPAHNGRETIYPNGTLLIQNVTHNDAGIYTLHVIKENLVNEEVTRQFYVFSEPPKPSITSNNFNPVENKDIVVLTCQPETQNTTYLWWVNNQSLLVSPRLLLSTDNRTLVLLSATKNDIGPYECEIQNPVGASRSDPVTLNVRYESVQASSPDLSAGTAVSIMIGVLAGMALI

SEQ ID NO: 13 - protein, 9 aa, Mus musculus
GYQKMKALL

SEQ ID NO: 14 - protein, 8 aa, Mus musculus
SPHQVFNL

SEQ ID NO: 15 - protein, 9 aa, Mus musculus
SPSYVYHQF

SEQ ID NO: 16 - protein, 11 aa, Mus musculus
LPQELPGLVVL

SEQ ID NO: 17 - protein, 11 aa, Mus musculus
MPHSLLPLVTF

SEQ ID NO: 18 - protein, 10 aa, Mus musculus
KYLSVQSGQL

SEQ ID NO: 19 - protein, 10 aa, Mus musculus
KPLKEAPLDL

SEQ ID NO: 20 - protein, 30 aa, Artificial Sequence
  TLR9i
QLRKLNLSFNYQKRVSFAHLSLAPSFGSLV

SEQ ID NO: 21 - protein, 30 aa, Artificial Sequence
  TLR9i
LRALDFSGNALGHMWAEGDLYLHFFQGLSG

SEQ ID NO: 22 - protein, 104 aa, Artificial Sequence [sequence omitted]

SEQ ID NO: 23 - protein, 187 aa, Artificial Sequence
  CTLA4 trap
LIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRN

SEQ ID NO: 24 - DNA, 57 bp, Artificial Sequence
  miR
cagggctggcctatgggacagatactataagtcatctcatcctggggctgggtctgc

SEQ ID NO: 25 - protein, 25 aa, Mus musculus
TPRGKIQAKKWSLVPFSIPVFDILQ

SEQ ID NO: 26 - protein, 25 aa, Mus musculus
KADVFHAYLSLLTQTRPVQSWLCDP

SEQ ID NO: 27 - protein, 25 aa, Mus musculus
NDEPDLDPVQELLYDLRSQCDAIRV

SEQ ID NO: 28 - protein, 25 aa, Mus musculus
VKEEDSLHWQRPEDVQKVKALSFYQ

SEQ ID NO: 29 - protein, 25 aa, Mus musculus
LREEASEILEEMRHKLRIGAIRFFA

SEQ ID NO: 30 - protein, 25 aa, Mus musculus
AAALQMIIAYAYRGNLAVNDSTVEQ

SEQ ID NO: 31 - protein, 25 aa, Mus musculus
DYTAAGFSSFQKLRLDLTSMQIITT

SEQ ID NO: 32 - protein, 25 aa, Mus musculus
ILDLLLLYKKHKAKKMTVPVRRHAY

(19) United States
(12) Patent Application Publication
(10) Pub. No.: US 2022/0370581 A1
(43) Pub. Date: Nov. 24, 2022

(54) VACCINE AND METHOD FOR TREATING CANCER

(71) Applicant: China Medical University, Taichung City (TW)

(72) Inventors: Kun-San Chao, Taichung City (TW); Kevin Chih-Yang Huang, Taichung City (TW); Shu-Fen Chiang, Taichung City (TW)

(73) Assignee: China Medical University, Taichung City (TW)

(21) Appl. No.: 17/746,112

(22) Filed: May 17, 2022

Related U.S. Application Data
(60) Provisional application No. 63/189,861, filed on May 18, 2021.
(60) Provisional application No. 63/308,568, filed on Feb. 10, 2022.

(51) Int. Cl. A61K 39/00; A61K 39/39; A61K 38/17; A61K 39/395; A61P 35/00; A61K 45/06; C12N 7/00; C12N 15/86

(52) U.S. Cl. CPC A61K 39/0011; A61K 39/39; A61K 38/1709; A61K 39/3955; A61P 35/00; A61K 45/06; C12N 7/00; C12N 15/86; A61K 2039/55561; A61K 2039/5158; A61K 2039/55538; A61K 2039/55522; C12N 2750/14122; C12N 2750/14134; C12N 2750/14142; C12N 2750/14152

(57) ABSTRACT

A vaccine including a vector and a transgene is provided. The transgene encodes a plurality of peptides and is packaged in the vector, in which the peptides in order include a secretion signal peptide, at least one tumor antigen, at least one co-inhibitory peptide and a toll-like receptor 9 (TLR9) antagonist.

### CROSS REFERENCE TO RELATED APPLICATIONS

This application claims the benefits of priority of U.S. Provisional Application No. 63/189,861, filed on May 18, 2021 and U.S. Provisional Application No. 63/308,568, filed on Feb. 10, 2022, the content of which are incorporated herein by reference.

### SEQUENCE LISTING

The sequence listing submitted via EFS, in compliance with 37 CFR § 1.52(e)(5), is incorporated herein by reference. The sequence listing text file submitted via EFS contains the file “CP-5231-US_SEQ_LIST”, created on May 16, 2022, which is 12,586 bytes in size.

### BACKGROUND

### Technical Field

The present disclosure relates to a vaccine and a method for treating cancer. More particularly, the present disclosure relates to a vaccine specific for tumor antigen and a method for treating cancer used thereof.

### Description of Related Art

The main therapeutic strategies for cancer therapy are radiotherapy, chemotherapy, targeted therapy and surgery. Even the advance in drug development and surgery techniques, the 5-year survival rate of late-stage cancer patients is still poor, suggesting that developing novel therapeutic strategies is urgent such as cancer immunotherapy. Immunotherapy depends on the reactivation of immune system to eliminate tumors such as immune checkpoint blockade, cell therapy and cancer vaccine. Although the clinical response of immune checkpoint blockade is promising in several malignancies, the application is limited by the status of DNA mismatch repair deficiency (10-15% cancer patients) and density of immune cell infiltration, indicating majority of cancer patients is not suitable for immune checkpoint blockade. Therefore, developing novel immunotherapy strategies such as neoantigen-based immunotherapy is critical.

Neoantigens are derived from the somatic mutations during cancer progression, which elicit tumor-specific immune response. With the breakthrough of next-generation sequencing technology, identifying personalized neoantigens to develop cancer vaccines to activate tumor-specific immune responses is feasible, while radiotherapy (RT) and chemotherapy (CT) cannot only increase tumor antigen release but also change tumor microenvironment to a more permissible one. It is anticipated that neoantigen-based cancer vaccine when combined with RT and immunogenic CT can potentially achieve sustainable disease control in those who refractory to the conventional treatments.

Moreover, monotherapy with neoantigens peptide vaccine alone was not effective enough in eliminating tumor and the majority of mutations differ from patient to patient, making neoantigens more personalized with high cost and long time. Therefore, targeting the frequently shared neoantigens and optimizing the delivery of neoantigen-based immunotherapy are good way to solve this problem.

### SUMMARY

According to one aspect of the present disclosure is to provide a vaccine including a vector and a transgene. The transgene encodes a plurality of peptides and is packaged in the vector, wherein the peptides in order include a secretion signal peptide, at least one tumor antigen, at least one co-inhibitory peptide and a toll-like receptor 9 (TLR9) antagonist. The at least one tumor antigen is a subtraction of tumor and normal cell antigens. The at least one co-inhibitory peptide includes programmed death-ligand 1 (PD-L1) antagonist, programmed cell death protein-1 (PD-1) antagonist or a cytotoxic T-lymphocyte-associated protein 4 (CTLA4) antagonist.

According to another aspect of the present disclosure is to provide a method for treating cancer. The method includes administering the vaccine according to the foregoing aspect to a subject in need for a treatment of cancer to induce an anti-tumor immune response in the subject.

According to still another aspect of the present disclosure is to provide a method for treating cancer by a cancer vaccine cocktail therapy including following steps. The vaccine according to the foregoing aspect is administered to a subject in need for a treatment of cancer to induce an immune priming against the at least one tumor antigen in the subject. An enhancer is administered to the subject to enhance local tumor control in the subject. A booster is administered to the subject to prevent local recurrence and metastasis in the subject.

### BRIEF DESCRIPTION OF THE DRAWINGS

The present disclosure can be more fully understood by reading the following detailed description of the embodiment, with reference made to the accompanying drawings as follows:

FIG. 1 is a schematic view showing a construction of a vaccine according to one embodiment of the present disclosure.

FIGS. 2A, 2B and 2C are schematic views showing mechanism of the vaccine delivery of a transgene into a subject and the interaction of the encoded peptides in the subject of the present disclosure.

FIG. 3A is a schematic view showing a construction of Example 1 of a neoAg peptide-based cancer vaccine.

FIG. 3B is a schematic view showing a treatment strategy of Example 1 of the neoAg peptide-based cancer vaccine combined with the radiotherapy in an animal treatment test.

FIG. 3C shows the analysis result of the effect of Example 1 of the neoAg peptide-based cancer vaccine in the treatment of a colorectal cancer.

FIGS. 4A, 4B, 4C, 4D and 4E show the analysis result of the effect of Example 1 of the neoAg peptide-based cancer vaccine on infiltration of immune cells for anti-tumor immunity.

FIG. 5A is a schematic view showing an experiment process of ex vivo immune analysis.

FIGS. 5B, 5C, 5D, 5E and 5F show the analysis results of the ex vivo immune analysis of Example 1 of the neoAg peptide-based cancer vaccine.

FIGS. 6A and 6B show the analysis results of the effect of Example 1 of the neoAg peptide-based cancer vaccine on infiltration of immune cells for anti-tumor immunity.

FIGS. 6C and 6D show the analysis results of the effect of Example 1 of the neoAg peptide-based cancer vaccine in tumor microenvironment (TME) after administering the radiotherapy.

FIG. 7A is a schematic view showing a construction and a treatment strategy of Examples 4, 6 and 8 of AAV-based cancer vaccines.

FIGS. 7B, 7C, 7D, 7E and 7F show the analysis results of the effect of Examples 4, 6 and 8 of the AAV-based cancer vaccines in the treatment of the colorectal cancer.

FIG. 8A is a schematic view showing a construction of Example 10 of a vaccine of the present disclosure.

FIG. 8B is a schematic view showing a treatment strategy of Example 10 of the vaccine of the present disclosure combined with the radiotherapy according to one example of the present disclosure in an animal treatment test.

FIG. 8C shows the analysis result of the effect of Example 10 of the vaccine of the present disclosure in the treatment of the colorectal cancer.

FIG. 9 shows the survival curve of colorectal cancer mice treated with Example 10 of the vaccine of the present disclosure.

FIG. 10A is a schematic view showing a construction and a treatment strategy of Example 13 of the vaccine of the present disclosure combined with the radiotherapy according to one example of the present disclosure in an animal treatment test.

FIGS. 10B, 10C, 10D, 10E, 10F and 10G show analysis results of the therapeutic effect of Example 13 of the vaccine of the present disclosure with the radiotherapy in the treatment of the colorectal cancer.

FIG. 11A is a schematic view showing a construction and a treatment strategy of Example 16 of the vaccine of the present disclosure combined with the radiotherapy according to one example of the present disclosure in an animal treatment test.

FIGS. 11B, 11C, 11D, 11E, 11F and 11G show the analysis results of the effect of Example 16 of the vaccine of the present disclosure in the treatment of mammary cancer.

FIG. 12A is a schematic view showing a treatment strategy of a method for treating cancer by a cancer vaccine cocktail therapy according to one embodiment of the present disclosure.

FIG. 12B is a schematic view showing a method for treating cancer by a cancer vaccine cocktail therapy according to one example of one embodiment of the present disclosure.

FIG. 13A is a schematic view showing a treatment strategy of a cancer vaccine cocktail therapy according to one example of the present disclosure in an animal treatment test.

FIGS. 13B, 13C and 13D show the analysis results of the therapeutic effect of the cancer vaccine cocktail therapy of the present disclosure in the treatment of the colorectal cancer.

### DETAILED DESCRIPTION

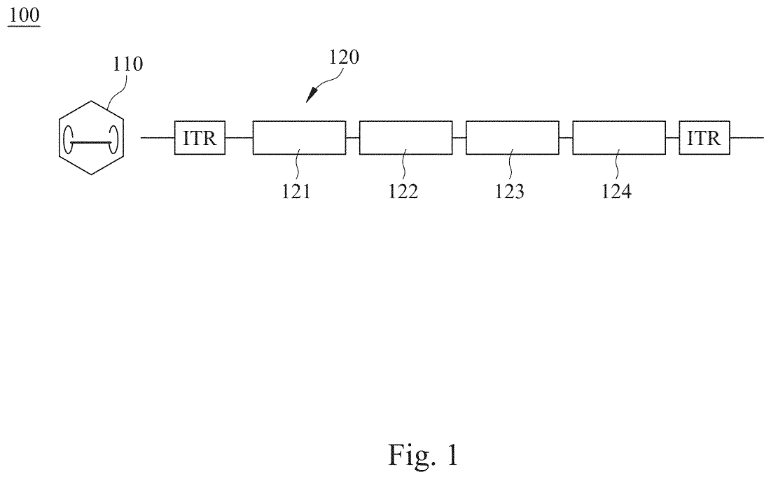

Please refer to FIG. 1, which is a schematic view showing a construction of a vaccine 100 according to one embodiment of the present disclosure. The vaccine 100 of the present disclosure includes a vector 110 and a transgene 120 packaged in the vector 110.

The vector 110 is for enhancing tumor antigens expression with diverse tropism, and can be a vaccinia viral vector, an adeno-associated virus (AAV) vector or a nanoparticle. Preferably, the AAV vector can be an adeno-associated virus 2 (AAV2) vector or an adeno-associated virus 6 (AAV6) vector. The nanoparticle can include but not limit to a liposome-derived delivery system [such as dicetyl phosphate-tetraethylenepentamine-based polycation liposome (TEPA-PCL), lipoplex (like DOTMA:cholesterol:TPGS lipoplex or DDAB:cholesterol:TPGS lipoplex), cationic liposome-hyaluronic acid (LPH) nanoparticle], a lipid nanoparticle (LNP), a polyethyleneimine (PEI) or PEI-conjugate, a dendrimer nanoparticle, a poly(amidoamine) (PAMAM) nanoparticle, poly(lactide-co-glycolide) (PLGA) nanoparticle, an atelocollagen nanoparticle and a silica nanoparticle.

The transgene 120 encodes a plurality of peptides, wherein the peptides in order include a secretion signal peptide 121, at least one tumor antigen 122, a co-inhibitory peptide 123 and a toll-like receptor 9 (TLR9) antagonist 124.

The secretion signal peptide 121 is for assisting tumor antigen secretion. Preferably, the secretion signal peptide 121 can be an interleukin 2 signal peptide (IL2 sp) or an interleukin 12 signal peptide (IL12 sp).

The at least one tumor antigen 122 is for increasing an anti-tumor immune response in a subject in need for a treatment of cancer, wherein is the at least one tumor antigen 122 is a subtraction of tumor and normal cell antigens. Preferably, the at least one tumor antigen 122 can be selected from a tumor-associated antigen (TAA), a tumor-specific antigen (TSA), an oncogenic mutation, an aberrantly expressed tumor-specific antigen (aeTSA) and a shared neoantigen (neoAg). In addition, the at least one tumor antigen 122 can be selected by comparing whole exome sequencing of matched tumor and normal cell DNA from the subject to identify tumor-specific somatic mutations (neoantigens), and then selecting polynucleotides encoding the neoantigens from a pre-existing library of neoantigen-encoding polynucleotides. The TAA was highly expressed on tumor cells with lower expression on normal cells. For example, but are not limited to, the TAA in breast cancer includes mammaglobin-A overexpressed in breast cancer, prostate specific antigen (PSA), melanoma antigen recognized by T cells (MART 1), melanocyte protein PMEL, Bcr/Abl tyrosin-kinase, HPVE6, E7, MZ2-E, MAGE-1 and MUC-1. The TSAs were found on cancer cells only, not on healthy cells. For example, but are not limited to, the TSA includes driver genes KRAS-G12/13 codon hotspot mutations, TP53 hotspot mutations, PIK3CA hotspot mutations, BRAF mutations and frameshift mutations. The aeTSA derives from aberrant expression of unmutated transcripts that are not expressed in any normal somatic cell, including medullary thymic epithelial cells (mTEC), which orchestrate central immune tolerance.

The co-inhibitory peptide 123 is for blocking the co-inhibitory signals in dendritic cell (DC) and increase antigen presentation ability of DC, wherein the co-inhibitory peptide 123 includes programmed death-ligand 1 (PD-L1) antagonist, programmed cell death protein-1 (PD-1) antagonist or a cytotoxic T-lymphocyte-associated protein 4 (CTLA4) antagonist. Preferably, the PD-L1 antagonist can include a PL-L1 trap and a PD-1 peptide, the PD-1 antagonist can include a PD-1 trap and a PD-L1/PD-L2 peptide, and the CTLA4 antagonist can include a CTLA4 trap and an antagonistic antibody against CTLA4.

The TLR9 antagonist 124 is an anti-viral clearance sequence for attenuating the innate immunity of viral clearance and maintain high antigen load. Preferably, the TLR9 antagonist 124 can be selected from a CpG oligonucleotide TLR9 binding domain, a TLR decoy peptide or a CpG binding sequence.

In addition, the vaccine 100 of the present disclosure can includes a co-stimulatory peptide for increasing the recruitment and activation of DC, wherein the co-stimulatory peptide is selected from a granulocyte-macrophage colony-stimulating factor (GM-CSF), interleukin 12 (IL12) and interferon (IFNs).

According to one embodiment, the method for treating cancer includes administering the vaccine according to the foregoing aspect to a subject in need for a treatment of cancer to induce an anti-tumor immune response in the subject. Preferably, the method for treating cancer in this embodiment can further include administering radiotherapy to the subject. In addition, the vaccine of the present disclosure can be under conditions wherein the peptides are expressed and synergistically promote a tumor-specific immune response in the subject, and synergistically prolong subject survival.

“Cancer” refers to a physiological condition in a mammal characterized by a disorder of cell growth. A “tumor” includes one or more cancer cells. Examples of cancer include, but are not limited to, carcinoma, lymphoma, blastoma, sarcoma, and leukemia or lymphoid malignancies. More specific examples of such cancers include breast cancer, colon cancer, rectal cancer, colorectal cancer, lung cancer including small cell lung cancer, non-small cell lung cancer (NSCLC), lung adenoma, and lung squamous cell carcinoma, squamous cell carcinoma (e.g., epithelial squamous cell carcinoma), peritoneal cancer, hepatocellular carcinoma, gastric cancer including gastrointestinal cancer, pancreatic cancer, glioblastoma, cervical cancer, ovarian cancer, liver cancer, bladder cancer, endometrial cancer or uterine cancer, salivary gland cancer, kidney cancer, prostate cancer, vulvar cancer, thyroid cancer, anal cancer, penile cancer, and head and neck cancer.

An “effective amount” refers to an amount of the vaccine of the present disclosure effective to “treat” a disease or disorder in a subject. The effective amount is to some extent related to the biological or medical response of the tissue, system, animal or human to whom it is administered, for example, when administered, it is sufficient to prevent the development of one or more diseases or conditions or to alleviate the symptoms of one or more conditions or conditions being treated to a certain extent. A therapeutically effective amount will vary depending on the disease and its severity, as well as the age and weight of the mammal to be treated.

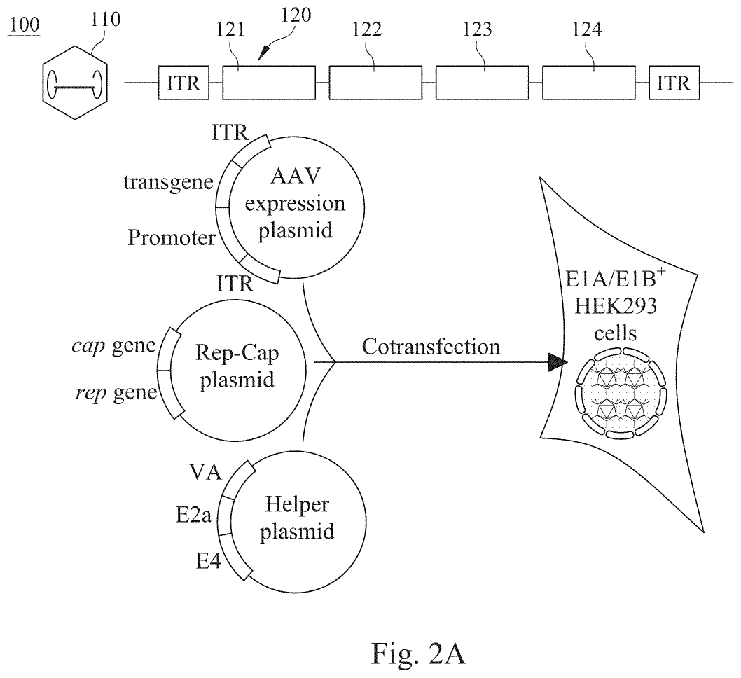

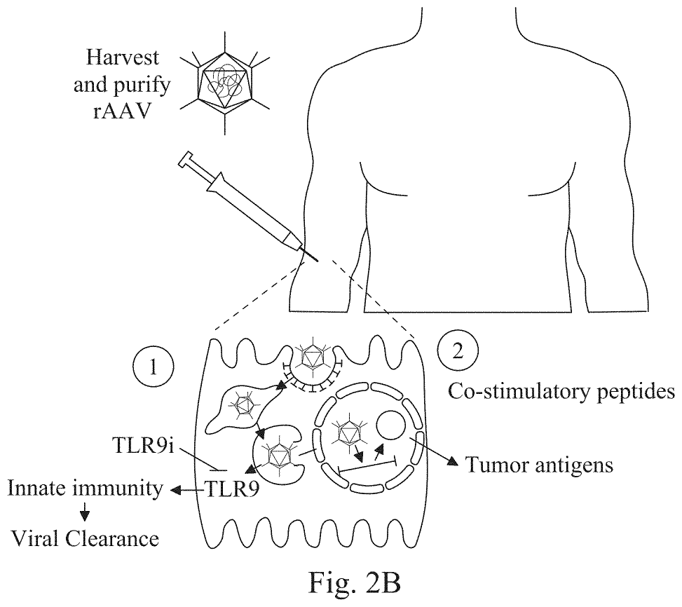

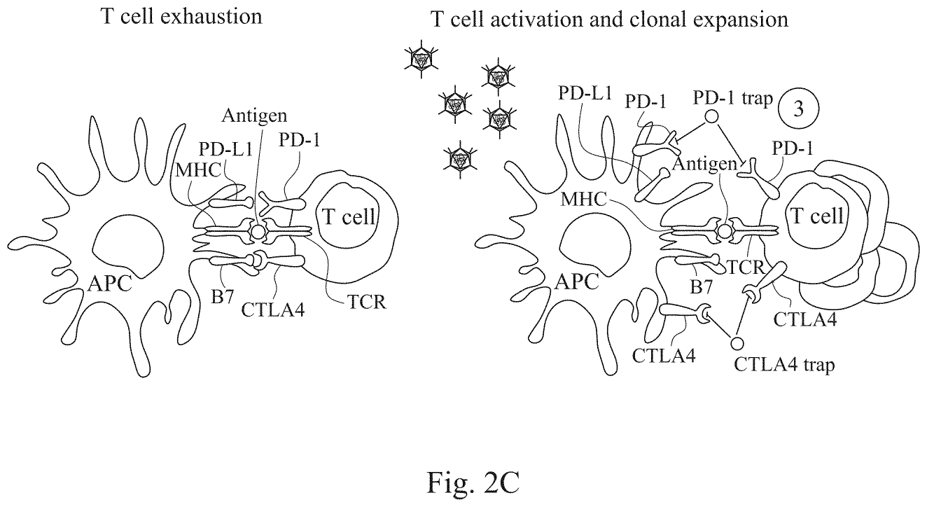

Please refer to FIGS. 2A, 2B and 2C, which are schematic views showing mechanism of the vaccine delivery of a transgene into a subject and the interaction of the encoded peptides in the subject of the present disclosure. The vaccine of the present disclosure can effectively suppress immune checkpoints, increase the amount of tumor antigens presented, and activate tumor immune responses.

Reference will now be made in detail to the present embodiments of the present disclosure, examples of which are illustrated in the accompanying drawings. However, following examples are for illustration only, but the present disclosure is not limited thereto. For example, the vector used in following examples is the AAV vector, but the vector is used to deliver the transgene to the target cell, so it is expected that other vectors, such as the vaccinia viral vector or the nanoparticle, can be used to achieve the same effect.

### EXAMPLES AND COMPARISONS

### Examples 1-3

First, we uncovered that the shared neoantigens (hereafter “neoAgs”) profiles in the residual tumors after chemotherapy (CT) and radiotherapy (RT), and established the neoAgs profiles within refractory and relapse tumors. These neoAgs can utilize to develop in vitro diagnosis (IVD) testing and antibody-based immunotherapy drugs. Moreover, these neoAgs can be the key ingredients for improving the tumor-specific of DC vaccine and DC-DIK cell therapy, and develop a neoAgs peptide-based cancer vaccine immunotherapy to improve the therapeutic efficacy of RT, CT and cell therapy. Please refer to Table 1, which is a list of neoAgs in mouse colon carcinoma CT26 cell line (hereinafter referred to as “CT26 cell”).

TABLE 1
The neoAgs in CT26 cell

| neoAg | Gene origin | neoAg sequence | T cell activation |
|---|---|---|---|
| 1 | E2f8 | SEQ ID NO: 1 | CD8 |
| 2 | Slc20a1 | SEQ ID NO: 2 | CD4 |
| 3 | Phf3 | SEQ ID NO: 3 | CD8 |
| 4 | Dhx35 | SEQ ID NO: 4 | CD4 |
| 5 | Mtch1 | SEQ ID NO: 5 | CD8 |
| 6 | Slc4a3 | SEQ ID NO: 6 | ND |
| 7 | Agx2l2 | SEQ ID NO: 7 | ND |
| 8 | Glud1 | SEQ ID NO: 8 | CD8 |

Further, we developed a neoAg peptide-based cancer vaccine including the aforementioned neoAgs to confirm the therapeutic effect on cancer treatment.

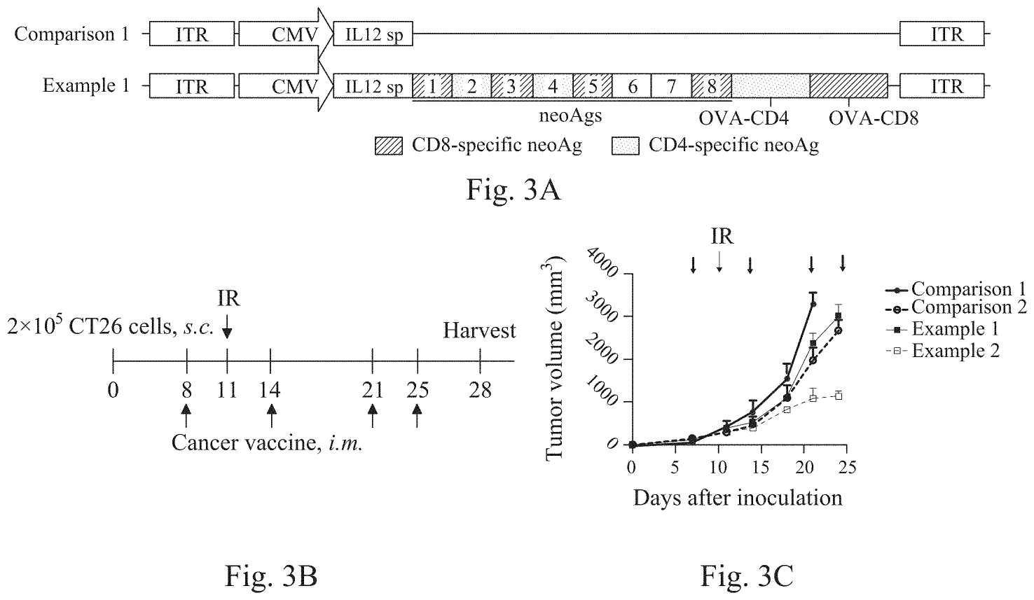

Please refer to 3A, which is a schematic view showing a construction of Example 1 of a neoAg peptide-based cancer vaccine. In FIG. 3A, the peptides encoded by the transgene in Example 1 of the neoAg peptide-based cancer vaccine includes an interleukin 12 signal peptide (IL12 sp), neoAgs and two ovalbumin sequences (OVA-CD4 and OVA-CD8), and corresponding nucleotide fragments of the peptides are cloned into a CMV-driven pAAV-CMV expression vector. The IL12 sp is to increase the neoAgs secretion, and the amino acid sequence of the ID 2 sp is referenced as SEQ ID NO: 11. The neoAgs includes the neoAgs 1-8 listed in Table 1 fused by RERK linkers. The OVA-CD4 and the OVA-CD8 are used as positive control, and the amino acid sequence of the OVA-CD4 and the OVA-CD8 is referenced as SEQ ID NO: 9 and SEQ ID NO: 10, respectively. In addition, Comparison 1 is an empty pAAV-CMV vector including the nucleotide fragments encoding the ID 2 sp but not including the nucleotide fragments encoding the neoAgs.

Please further refer to FIG. 3B and Table 2. FIG. 3B is a schematic view showing a treatment strategy of Example 1 of the neoAg peptide-based cancer vaccine combined with the radiotherapy in an animal treatment test. Table 2 shows the treatment strategy of Examples 1-2 and Comparisons 1-2.

TABLE 2
The treatment strategy of Examples 1-2 and Comparisons 1-2

| Group | Cancer vaccine | Radiotherapy |
|---|---|---|
| Comparison 1 | Comparison 1 | — |
| Comparison 2 | Comparison 1 | 5 Gy × 1 |
| Example 1 | Example 1 | — |
| Example 2 | Example 1 | 5 Gy × 1 |

To demonstrate the therapeutic efficacy of the Example 1 of the neoAg peptide-based cancer vaccine, a colorectal cancer mouse model is established first. Six-week-old female BALB/c mice were inoculated subcutaneously 2×10⁵ CT26 cells with 20% matrigel (Corning, Union City, Calif., USA) into the lower right leg. After 8 days, the colorectal cancer mice were randomly assigned into different groups, Example 1 and Comparison 1 (1×10⁸ vg) were administered via intramuscular injection every 6 days for 3 times and boost the 4th times on Day 25. For radiotherapy, colorectal cancer mice after complete anesthesia were placed the right leg in the irradiation field, we give one treatment, the local tumors were received 5 Gy fractionated radiotherapy on Day 11. At the same time, the tumor volume was measured and calculated by the formula: V=(L×W²)/2 every 3 days until Day 28 sacrificed. The tumor tissues were collected for following immune analysis.

Please refer to FIG. 3C, which show the analysis result of the effect of Example 1 of the neoAg peptide-based cancer vaccine in the treatment of the colorectal cancer. In FIG. 3C, compared with Comparison 1, Example 1 treated with Example 1 of the neoAg peptide-based cancer vaccine alone can significantly inhibit tumor growth, which can achieve similar effect as Comparison 2 (treated with radiotherapy alone). Example 2 treated with Example 1 of the neoAg peptide-based cancer vaccine and radiotherapy at the same time had a more significant effect of inhibiting tumor growth. The result indicates that Example 1 of the neoAg peptide-based cancer vaccine increases the therapeutic efficacy of the radiotherapy.

To demonstrate the effect of the Example 1 of the neoAg peptide-based cancer vaccine on infiltration of immune cells for anti-tumor immunity, isolation of tumor-infiltrating lymphocytes was performed. Isolated fresh tumors from colorectal cancer mice of Example 1, Example 2, Comparison 1 and Comparison 2, place the tumor in a 6 cm dish containing 5 ml of RPMI 1640 media at room temperature, then mince the tumor into 1-2 mm small pieces using sterile blade. Prepare a 50 ml conical tube, place a 70 μm cell strainer in the top, and transfer all the tumor tissue to the strainer by sterile dropper. If there are pieces of tissue left on, use the rubber of 5 mL syringe and add another RPMI 1640 media to mesh the tissue through the strainer. Carefully transfer all the cell solution into the 15 mL conical tube containing Ficoll-Paque at the bottom. Centrifuge at 1025× g for 20 mins at 20° C. with slow acceleration and brakes turned off. Carefully transfer the layer of mononuclear cells to a new 50 ml conical tube using a sterile pipet, add 10 mL RPMI 1640 media then centrifuge at 650× g for 10 mins at 20° C. Remove the supernatant, and gently resuspend cells in 10 ml of complete RPMI 1640 media, centrifuge at 650× g for 10 mins at 20° C. again. Remove the supernatant and add 1 mL RPMI 1640 media resuspend, these are the extracted tumor-infiltrating lymphocytes (TILs).

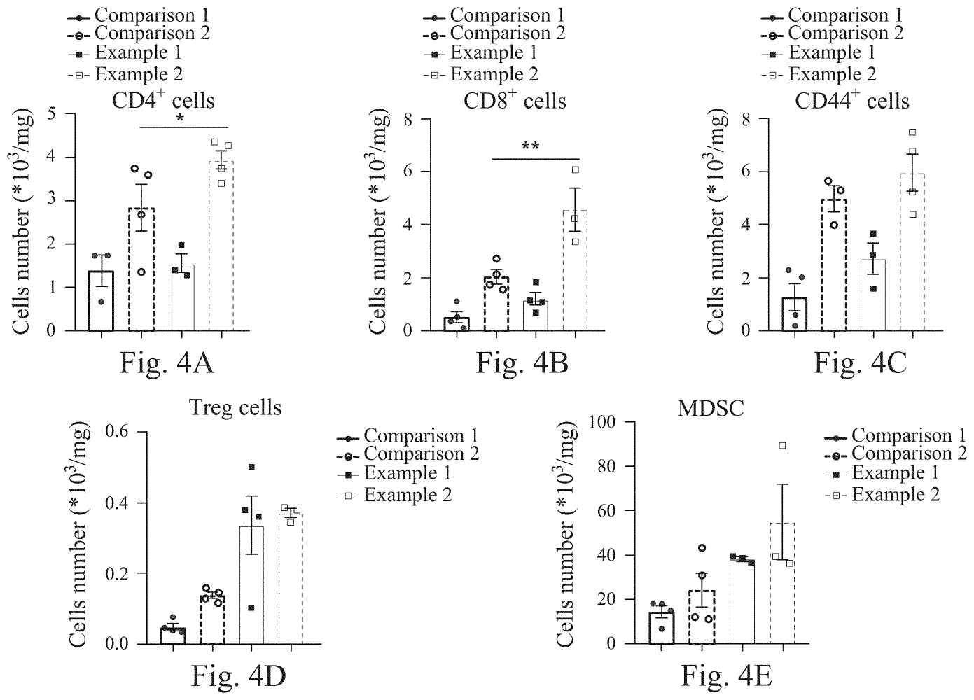

Please refer to FIGS. 4A, 4B, 4C, 4D and 4E, which show the analysis result of the effect of Example 1 of the neoAg peptide-based cancer vaccine on infiltration of immune cells for anti-tumor immunity, wherein * represents p<0.05, and ** represents p<0.01 using one-way ANOVA. In FIGS. 4A to 4E, compared with Comparison 1, Comparison 2 and Example 1, Example 2 treated with Example 1 of the neoAg peptide-based cancer vaccine and radiotherapy at the same time can significantly increase cells number of CD4⁺ cells, CD8⁺ cells, CD44⁺ cells, Treg cells and Myeloid-derived suppressor cells (MDSC), wherein the cells number of CD4⁺ cells represents helper T lymphocyte (Th) response, the cells number of CD8⁺ cells represents cytotoxic T lymphocyte (CTL) response, the cells number of CD44⁺ cells represents effector/memory T cell response, and the cells number of Treg cells and MDSC represent immune inhibitory cells response. The result indicates that Example 1 of the neoAg peptide-based cancer vaccine promotes infiltration of immune cells for anti-tumor immunity.

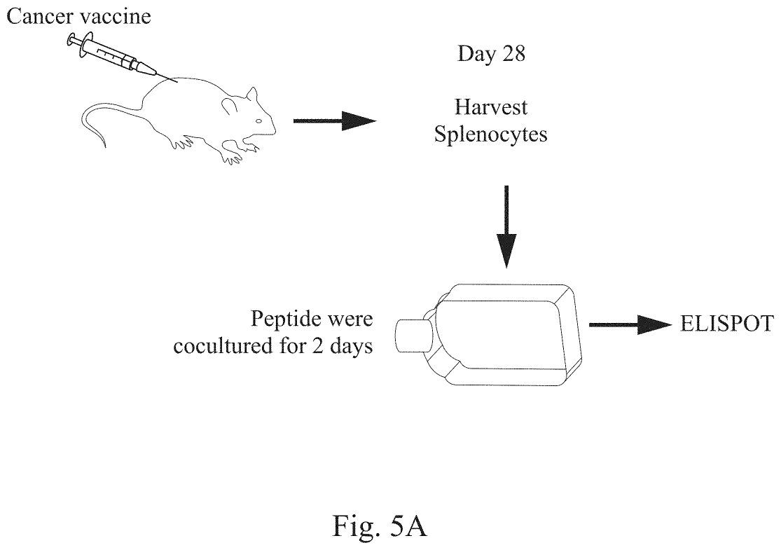

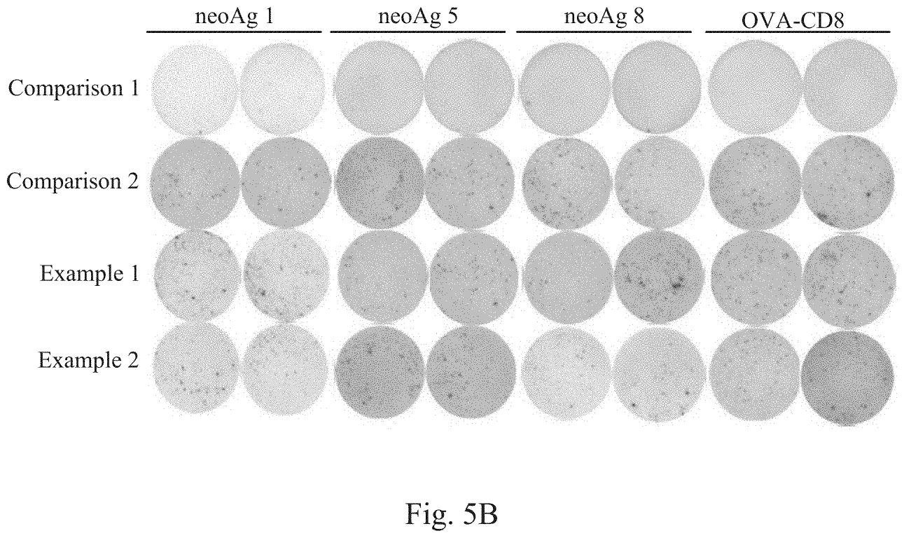

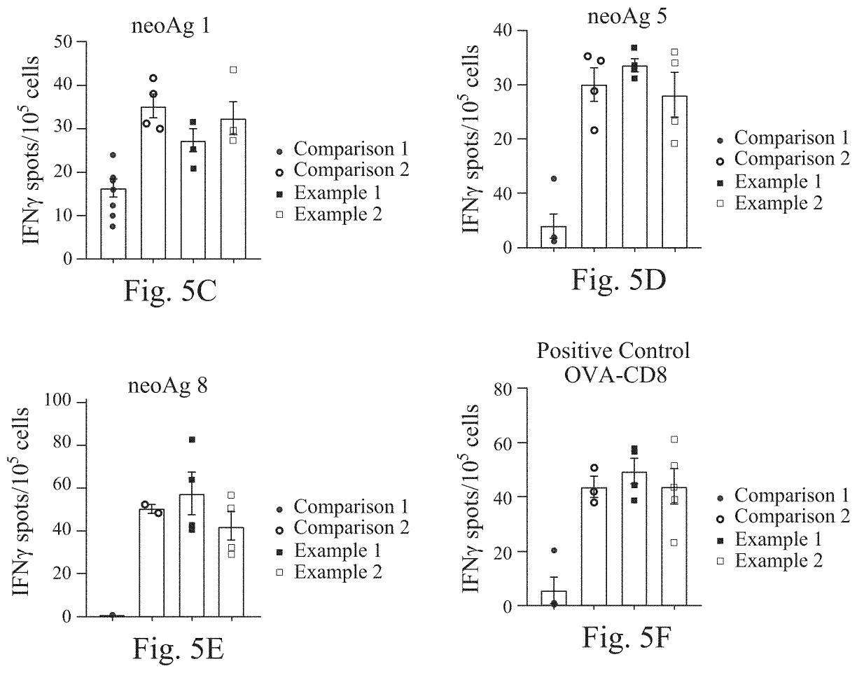

Please refer to FIGS. 5A, 5B, 5C, 5D, 5E and 5F. FIG. 5A is a schematic view showing an experiment process of ex vivo immune analysis. FIGS. 5B, 5C, 5D, 5E and 5F show the analysis results of the ex vivo immune analysis of Example 1 of the neoAg peptide-based cancer vaccine. For the ex vivo immune analysis, IFNγ ELISpot assays kit (Abcam) were performed on single-cell suspensions of colorectal cancer mice spleens. Seed 2.5×10⁵ splenocytes per 96 well in complete RPMI 1640 supplemented with 2 mM L-glutamine, 0.5 ug/mL concavalin A and 2 ng/mL m-IL2, and then incubate 2 days. Remove non adherent materials, and then replace the culture media supplemented with peptides 1 μg/mL stimulation for 24 hrs. The positive control was added 1 ng/mL PMA and 500 ng/mL Inomycin in RPMI 1640 media. Finally, qualitative measurement is performed to detect the spot of IFNγ production and secretion. In FIGS. 5B to 5F, the results indicate that Example 1 of the neoAg peptide-based cancer vaccine induces neoAg-specific CD8⁺ T cell response.

To demonstrate the effect of the Example 1 of the neoAg peptide-based cancer vaccine in tumor microenvironment (TME), isolation of tumor-infiltrating lymphocytes was performed. Colorectal cancer mice were randomly assigned into different groups, Example 1 and Comparison 1 (1×10⁸ vg) and PBS were administered via intramuscular injection 4 times on Day 8, Day 14, Day 21 and Day 25, respectively. For radiotherapy, colorectal cancer mice after complete anesthesia were placed the right leg in the irradiation field, the local tumors were received 5 Gy fractionated radiotherapy twice on Day 11 and Day 18. The colorectal cancer mice were sacrificed on Day 28, and the tumor tissues were collected for immune analysis. The treatment strategy of Examples 1, 3, Comparisons 1, 3 and Controls 1, 3 are shown in Table 3.

TABLE 3
The treatment strategy of Examples 1,
3, Comparisons 1, 3 and Controls 1, 3

| Group | Cancer vaccine | Radiotherapy |
|---|---|---|
| Control 1 | PBS | — |
| Control 3 | PBS | 5 Gy × 2 |
| Comparison 1 | Comparison 1 | — |
| Comparison 3 | Comparison 1 | 5 Gy × 2 |
| Example 1 | Example 1 | — |
| Example 3 | Example 1 | 5 Gy × 2 |

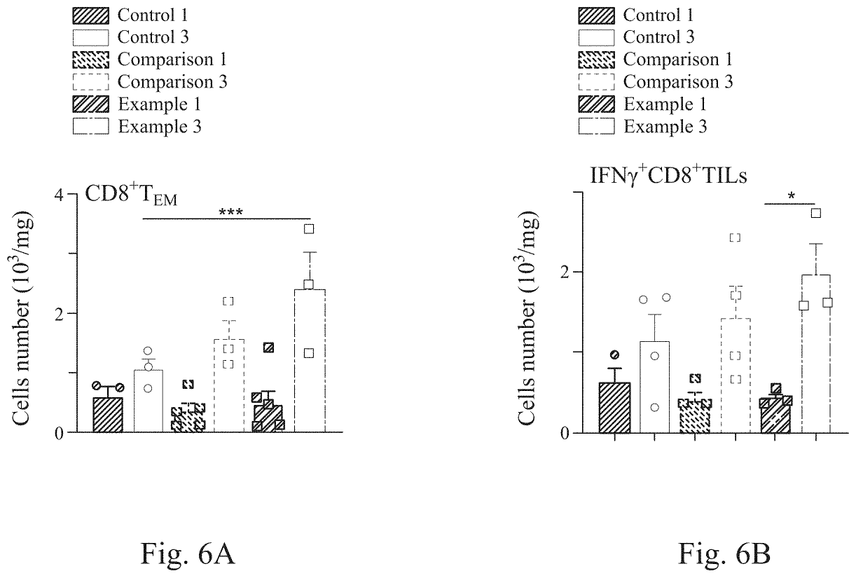

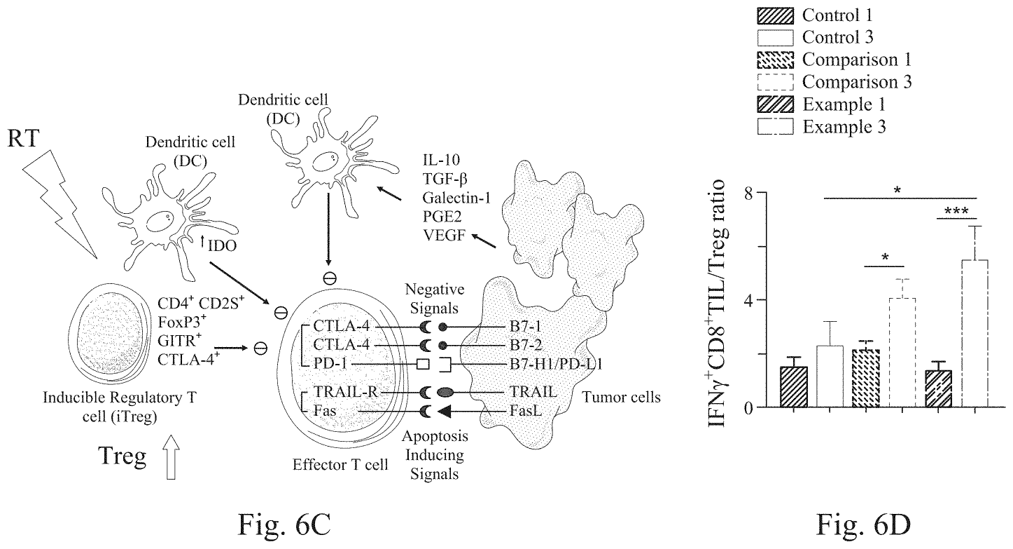

Please refer to FIGS. 6A, 6B, 6C and 6D. FIGS. 6A and 6B show the analysis results of the effect of Example 1 of the neoAg peptide-based cancer vaccine on infiltration of immune cells for anti-tumor immunity. FIGS. 6C and 6D show the analysis results of the effect of Example 1 of the neoAg peptide-based cancer vaccine in tumor microenvironment (TME) after administering the radiotherapy. In FIGS. 6A, 6B and 6D, * represents p<0.05, and *** represents p<0.001 using one-way ANOVA. In FIGS. 6A to 6D, compared with Controls 1 and 3, Comparison 1, Comparison 3 and Example 1, Example 3 treated with Example 1 of the neoAg peptide-based cancer vaccine and radiotherapy at the same time significantly increases the cell number of CD8⁺T_{EM} and IFNγ⁺CD8⁺TILs and IFNγ⁺CD8⁺TIL/Treg ratio. The results show indicate the radiotherapy increases tumor-infiltrating effector/memory and cytotoxic CD8⁺ T cells and Example 1 of the neoAg peptide-based cancer vaccine reverses immunosuppressive state in TME after administering the radiotherapy.

### Examples 4-9

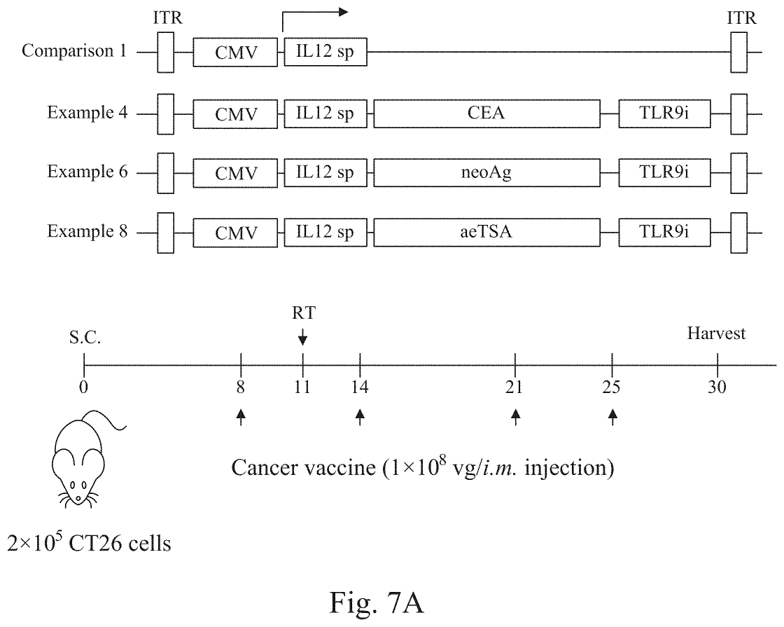

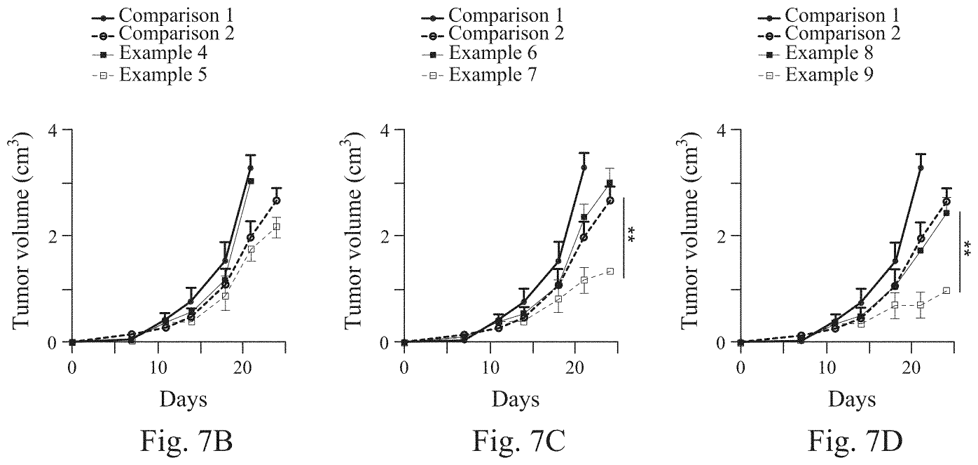

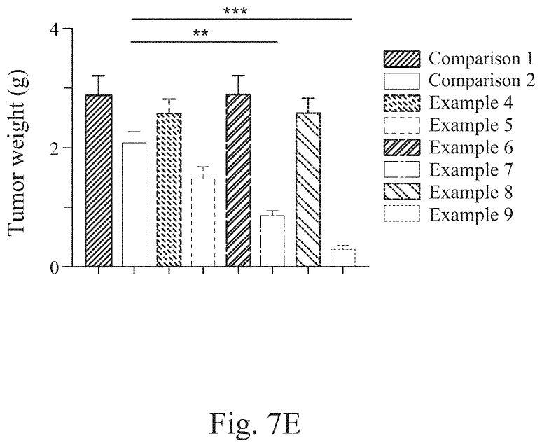

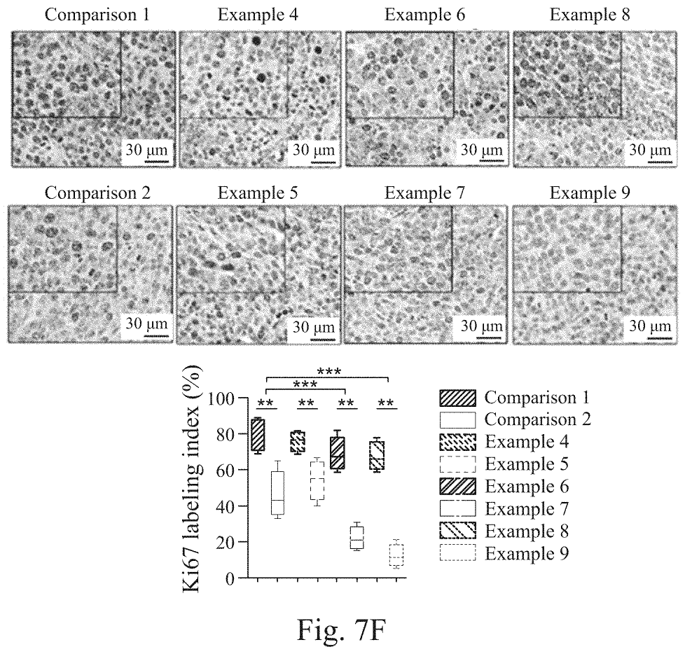

Further, we developed AAV-based cancer vaccines including the TLR9 antagonist and different tumor antigens to confirm the therapeutic effect on cancer treatment. Please refer to FIG. 7A, which is a schematic view showing a construction and a treatment strategy of Examples 4, 6 and 8 of AAV-based cancer vaccines.

In FIG. 7A, three AAV-based cancer vaccines (Example 4, Example 6 and Example 8) are engineered by inserting two short TLR9-inhibitory sequences (presents as “TLR9i” in FIG. 7A) into the pAAV-CMV vector including the nucleotide fragments encoding the IL12 sp to evade innate immunity for viral clearance and extend antigen expression. The peptides encoded by the transgene in Example 4 of the AAV-based cancer vaccine includes TAA carcinoembryonic antigen (CEA) as the at least one tumor antigen, and the amino acid sequence of the CEA is referenced as SEQ ID NO: 12. The peptides encoded by the transgene in Example 6 of the AAV-based cancer vaccine includes the neoAgs 1-8 (presents as “neoAg” in FIG. 7A) listed in Table 1 fused by RERK linkers as the at least one tumor antigen. The peptides encoded by the transgene in Example 8 of the AAV-based cancer vaccine includes aberrantly expressed tumor-specific antigens 1-7 (presents as “aeTSA” in FIG. 7A) listed in Table 4 as the at least one tumor antigen, wherein ERE is abbreviation for endogenous retroelement. The amino acid sequence of the two short TLR9-inhibitory sequences is referenced as SEQ ID NO: 20 and SEQ ID NO: 21, respectively. In addition, Comparison 1 is an empty pAAV-CMV vector including the nucleotide fragments encoding the IL12 sp but not including the nucleotide fragments encoding the tumor antigen.

TABLE 4
The aeTSAs in CT26 cell

| aeTSA | Gene origin | aeTSA sequence |
|---|---|---|
| 1 | ERE | SEQ ID NO: 13 |
| 2 | ERE | SEQ ID NO: 14 |
| 3 | ERE | SEQ ID NO: 15 |
| 4 | ERE | SEQ ID NO: 16 |
| 5 | intron | SEQ ID NO: 17 |
| 6 | coding exon in-frame | SEQ ID NO: 18 |
| 7 | intron | SEQ ID NO: 19 |

To demonstrate the therapeutic efficacy of the Examples 4, 6 and 8 of the AAV-based cancer vaccines, colorectal cancer mice were randomly assigned into different groups. Examples 4, 6 and 8 of the AAV-based cancer vaccines and Comparison 1 (1×10⁸ vg) were administered via intramuscular injection 4 times on Day 8, Day 14, Day 21 and Day 25, respectively. For radiotherapy, colorectal cancer mice after complete anesthesia were placed the right leg in the irradiation field, the local tumors were received 5 Gy fractionated radiotherapy on Day 11. The tumor volume was measured and calculated by the formula: V=(L×W²)/2 every 3 days until Day 30 sacrificed, and the tumor tissues were collected for immune analysis. The treatment strategy of Examples 4-9 and Comparisons 1-2 are shown in Table 5.

TABLE 5
The treatment strategy of Examples 4-9 and Comparisons 1-2

| Group | Cancer vaccine | Radiotherapy |
|---|---|---|
| Comparison 1 | Comparison 1 | — |
| Comparison 2 | Comparison 1 | 5 Gy × 1 |
| Example 4 | Example 4 | — |
| Example 5 | Example 4 | 5 Gy × 1 |
| Example 6 | Example 6 | — |
| Example 7 | Example 6 | 5 Gy × 1 |
| Example 8 | Example 8 | — |
| Example 9 | Example 8 | 5 Gy × 1 |

Please refer to FIGS. 7B, 7C, 7D, 7E and 7F, which show the analysis results of the effect of Examples 4, 6 and 8 of the AAV-based cancer vaccines in the treatment of the colorectal cancer, wherein ** represents p<0.01 and *** represents p<0.001 using one-way ANOVA. In FIGS. 7B to 7E, Example 4 of the AAV-based cancer vaccine alone did not protect colorectal cancer mice from tumor development, but Examples 6 and 8 of the AAV-based cancer vaccines monotherapy slightly delayed tumor growth. However, the groups (Examples 5, 7 and 9) treated with AAV-based cancer vaccine and radiotherapy at the same time had significant effects of inhibiting tumor growth. In FIG. 7F, the proliferating cell marker Ki67 was markedly decreased in Example 7 and Example 9. The results indicate that Examples 4, 6 and 8 of the AAV-based cancer vaccines significantly increases the therapeutic efficacy of radiotherapy and elicits a tumor antigen-specific immune response to delay tumor growth.

### Examples 10-12

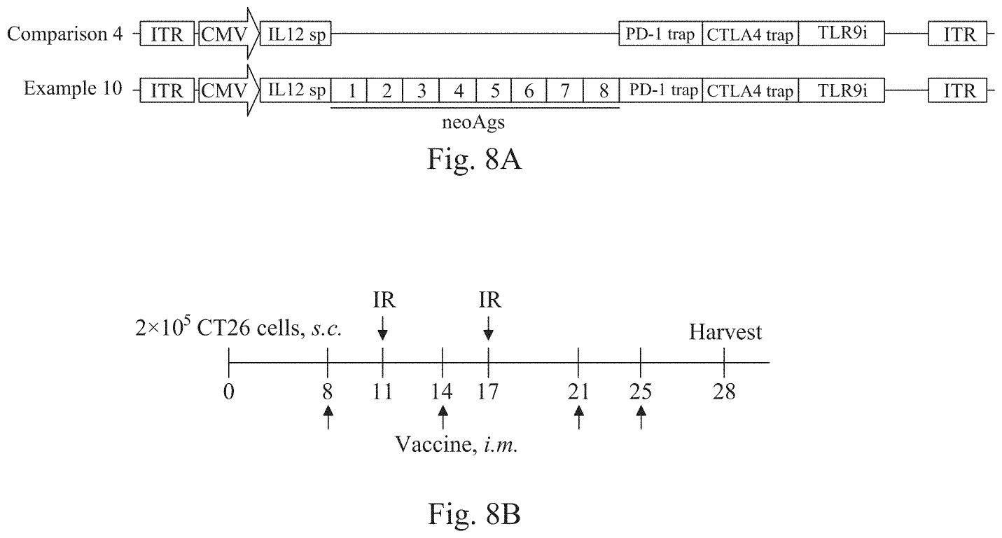

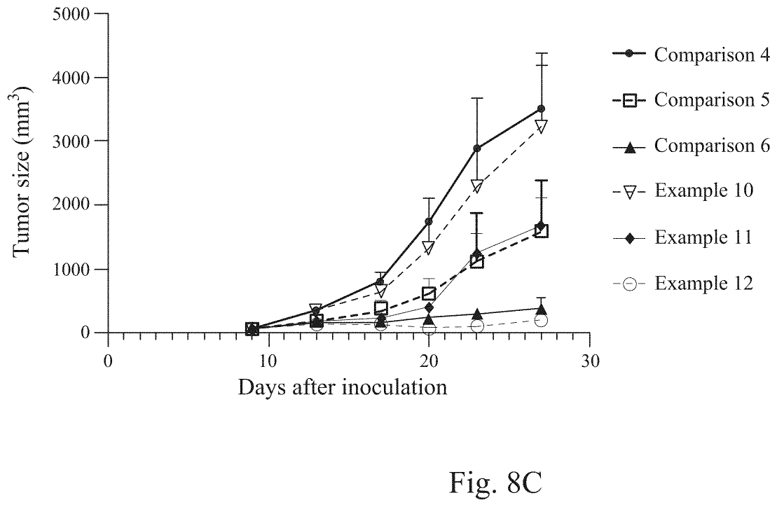

Further, we developed a vaccine of the present disclosure to confirm the therapeutic effect on cancer treatment. Please refer to FIG. 8A, which is a schematic view showing a construction of Example 10 of the vaccine of the present disclosure. In FIG. 8A, the peptides encoded by the transgene in Example 10 of the vaccine of the present disclosure includes the ID 2 sp as the secretion signal peptide, the neoAgs 1-8 listed in Table 1 as the at least one tumor antigen, a PD-1 trap and a CTLA4 trap as the least one co-inhibitory peptide, and the TLR9i as the TLR9 antagonist, and corresponding nucleotide fragments of the peptides are cloned into a CMV-driven pAAV-CMV expression vector. The amino acid sequence of the IL12 sp is referenced as SEQ ID NO: 11. The amino acid sequence of the PD-1 trap and the CTLA4 trap is referenced as SEQ ID NO: 22 and SEQ ID NO: 23, respectively. The TLR9i includes the amino acid sequences of SEQ ID NO: 20 and SEQ ID NO: 21. In addition, Comparison 4 is a pAAV-CMV vector including the nucleotide fragments encoding the IL12 sp, the PD-1 trap, the CTLA4 trap, and the TLR9i but not including the nucleotide fragments encoding the tumor antigen.

Please refer to FIG. 8B and Table 6. FIG. 8B is a schematic view showing a treatment strategy of Example 10 of the vaccine of the present disclosure combined with the radiotherapy according to one example of the present disclosure in an animal treatment test. Table 6 shows the treatment strategy of Examples 10-12 and Comparisons 4-6.

TABLE 6
The treatment strategy of Examples 10-12 and Comparisons 4-6

| Group | Vaccine | Radiotherapy |
|---|---|---|
| Comparison 4 | Comparison 4 | — |
| Comparison 5 | Comparison 4 | 5 Gy × 1 |
| Comparison 6 | Comparison 4 | 5 Gy × 2 |
| Example 10 | Example 10 | — |
| Example 11 | Example 10 | 5 Gy × 1 |
| Example 12 | Example 10 | 5 Gy × 2 |

To demonstrate the therapeutic efficacy of the Example 10 of the vaccine of the present disclosure, colorectal cancer mice were randomly assigned into different groups. Example 10 of the vaccine of the present disclosure and Comparison 4 (1×10⁸ vg) were administered via intramuscular injection 4 times on Day 8, Day 14, Day 21 and Day 25, respectively. For radiotherapy, colorectal cancer mice after complete anesthesia were placed the right leg in the irradiation field, the local tumors were received 5 Gy fractionated radiotherapy once on Day 11 or twice on Day 11 and Day 17. At the same time, the tumor volume was measured and calculated by the formula: V=(L×W²)/2 every 3 days until Day 28 sacrificed.

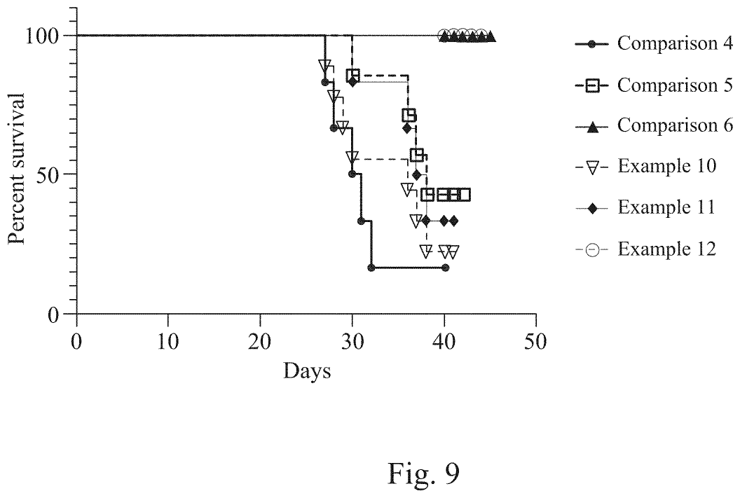

Please refer to FIGS. 8C and 9 and Table 7. FIG. 8C shows the analysis result of the effect of Example 10 of the vaccine of the present disclosure in the treatment of the colorectal cancer. FIG. 9 shows the survival curve of colorectal cancer mice treated with Example 10 of the vaccine of the present disclosure. Table 7 shows the complete response (CR) rate of Examples 10-12 and Comparisons 4-6.

TABLE 7
The CR rate of Examples 10-12 and Comparisons 4-6

| Group | CR rate |
|---|---|
| Comparison 4 | 0/6 |
| Comparison 5 | 0/6 |
| Comparison 6 | 0/6 |
| Example 10 | 1/6 |
| Example 11 | 0/6 |
| Example 12 | 2/5 |

In FIG. 8C, compared with other groups, Example 12 (administrated with Example 10 of the vaccine of the present disclosure and radiotherapy at the same time) significantly decreased tumor volume; it indicates that Example 10 of the vaccine of the present disclosure significantly promotes the therapeutic efficacy of the radiotherapy. In FIG. 9 and Table 7, 40% of colorectal cancer mice achieved a complete response (2/5) after treatment with Example 10 of the vaccine of the present disclosure; it indicates that Example 10 of the vaccine of the present disclosure significantly prolongs the survival time in vivo.

### Examples 13-15

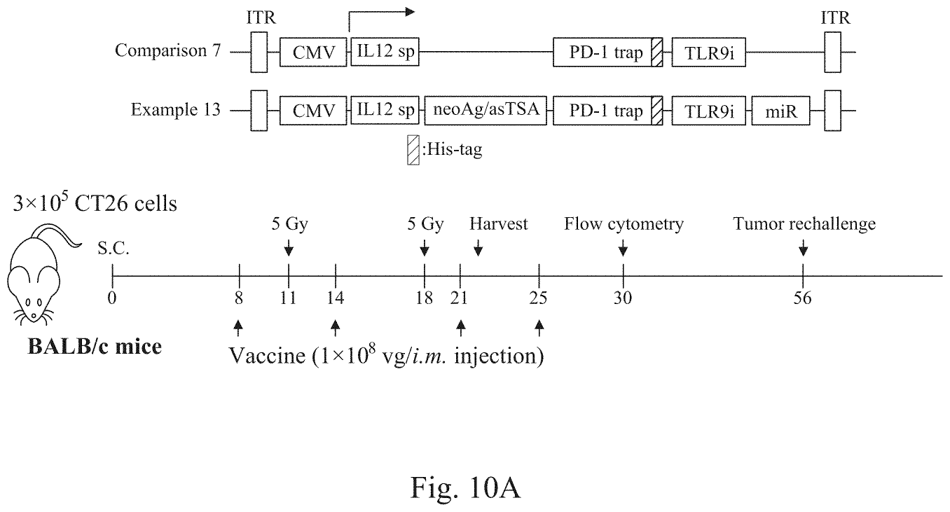

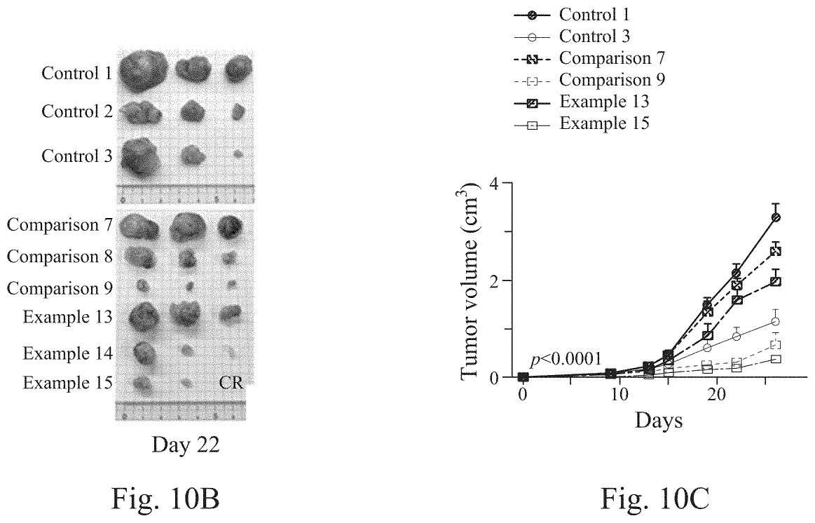

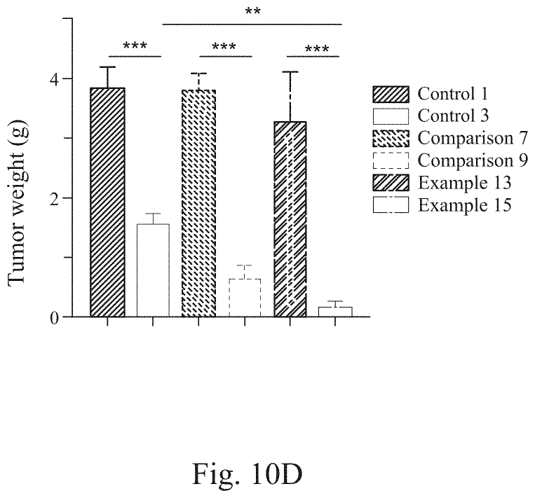

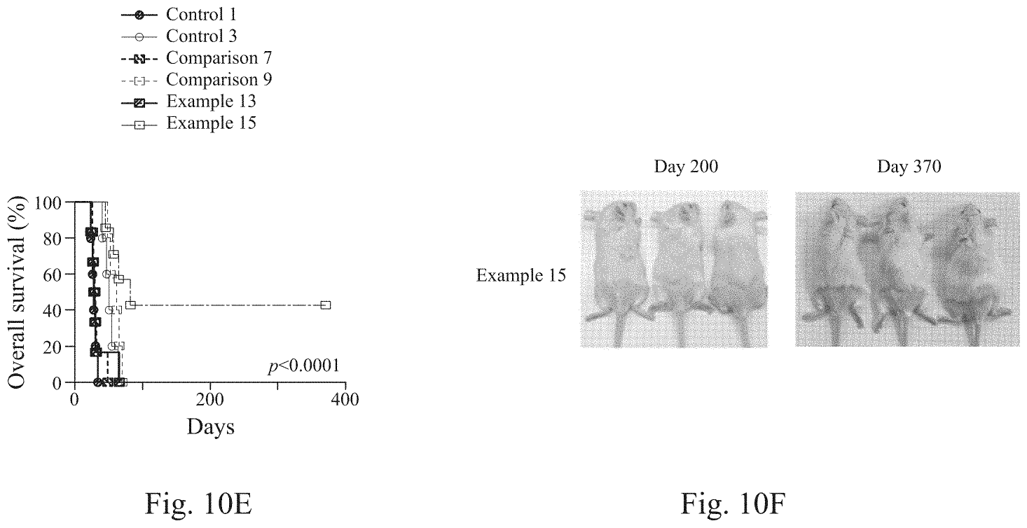

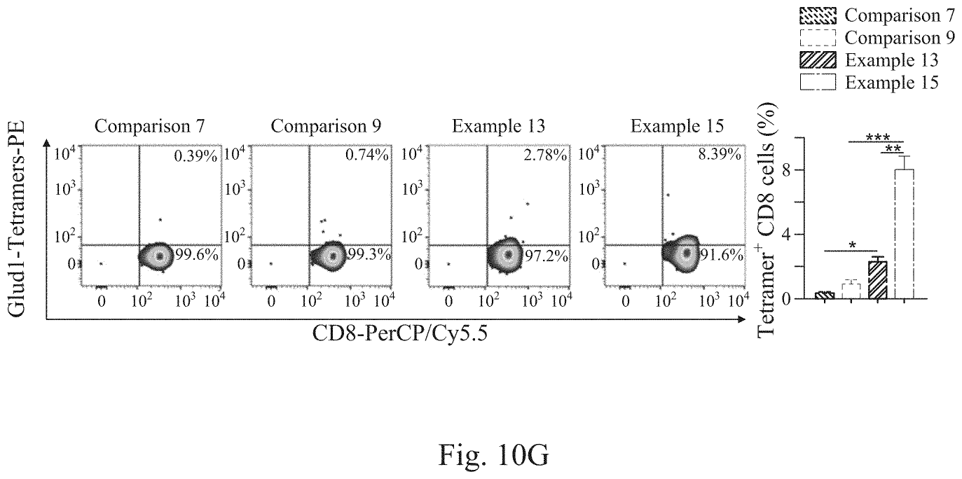

Further, we developed another vaccine of the present disclosure to confirm the therapeutic effect on cancer treatment. Please refer to FIG. 10A and Table 8. FIG. 10A is a schematic view showing a construction and a treatment strategy of Example 13 of the vaccine of the present disclosure combined with the radiotherapy according to one example of the present disclosure in an animal treatment test. Table 8 shows the treatment strategy of Examples 13-15, Comparisons 7-9 and Controls 1-3.

TABLE 8
The treatment strategy of Examples 13-
15, Comparisons 7-9 and Controls 1-3

| Group | Vaccine | Radiotherapy |
|---|---|---|
| Control 1 | PBS | — |
| Control 2 | PBS | 5 Gy × 1 |
| Control 3 | PBS | 5 Gy × 2 |
| Comparison 7 | Comparison 7 | — |
| Comparison 8 | Comparison 7 | 5 Gy × 1 |
| Comparison 9 | Comparison 7 | 5 Gy × 2 |
| Example 13 | Example 13 | — |
| Example 14 | Example 13 | 5 Gy × 1 |
| Example 15 | Example 13 | 5 Gy × 2 |

In FIG. 10A, the peptides encoded by the transgene in Example 13 of the vaccine of the present disclosure includes the IL12 sp as the secretion signal peptide, a neoAg/asTSA as the at least one tumor antigen, a PD-1 trap and a PD-L1 miRNA (presents as “miR” in FIG. 10A) as the least one co-inhibitory peptide, and the TLR9i as the TLR9 antagonist, and corresponding nucleotide fragments of the peptides are cloned into a CMV-driven pAAV-CMV expression vector. The amino acid sequence of the IL12 sp is referenced as SEQ ID NO: 11. The neoAg/asTSA includes the neoAgs 1-8 listed in Table 1 and the asTSAs 1-7 listed in Table 4. The amino acid sequence of the PD-1 trap is referenced as SEQ ID NO: 22, and the nucleic acid sequence of the PD-L1 miRNA is referenced as SEQ ID NO: 24. The TLR9i includes the amino acid sequences of SEQ ID NO: 20 and SEQ ID NO: 21. In addition, Comparison 7 is a pAAV-CMV vector including the nucleotide fragments encoding the IL12 sp, the PD-1 trap and the TLR9i but not including the nucleotide fragments encoding the tumor antigen and PD-L1 miRNA.

To demonstrate the therapeutic efficacy of the Example 13 of the vaccine of the present disclosure, colorectal cancer mice were randomly assigned into different groups. Example 13 of the vaccine of the present disclosure, Comparison 7 (1×10⁸ vg) and PBS were administered via intramuscular injection 4 times on Day 8, Day 14, Day 21 and Day 25, respectively. For radiotherapy, colorectal cancer mice after complete anesthesia were placed the right leg in the irradiation field, the local tumors were received 5 Gy fractionated radiotherapy once on Day 11 or twice on Day 11 and Day 18. In addition, colorectal cancer mice were inoculated subcutaneously 3×10⁵ CT26 cells with 20% matrigel for tumor rechallenge on Day 56. At the same time, the tumor volume was measured and calculated by the formula: V=(L×W²)/2 every 3 days, and flow cytometry was performed on Day 30. In addition, after intramuscular injection immunization with Example 13 of the vaccine of the present disclosure and Comparison 7, the levels of Glud1⁺ CD8 cells were measured in the blood of colorectal cancer mice by using a Glud1/MHC-I-specific tetramer assay.

Please refer to FIGS. 10B, 10C, 10D, 10E, 10F, 10G and Tables 9 and 10. FIGS. 10B, 10C, 10D, 10E, 10F and 10G show analysis results of the therapeutic effect of Example 13 of the vaccine of the present disclosure with the radiotherapy in the treatment of the colorectal cancer, wherein * represents p<0.05, ** represents p<0.01 and represents p<0.001 using one-way ANOVA. Table 9 shows the CR rate of Examples 13 and 15, Comparisons 7 and 9 and Controls 1 and 3, and Table 10 shows the median survival time of Examples 13 and 15, Comparisons 7 and 9 and Control 1 and 3.

TABLE 9
The CR rate of Examples 13 and 15, Comparisons
7 and 9 and Controls 1 and 3

| Group | CR rate |
|---|---|
| Control 1 | 0/6 |
| Control 3 | 0/6 |
| Comparison 7 | 0/6 |
| Comparison 9 | 0/6 |
| Example 13 | 0/6 |
| Example 15 | 3/7 |

TABLE 10
The median survival time of Examples 13 and
15, Comparisons 7 and 9 and Controls 1 and 3

| Group | median survival time |
|---|---|
| Control 1 | 28 |
| Control 3 | 51 |
| Comparison 7 | 30.5 |
| Comparison 9 | 62 |
| Example 13 | 28.5 |
| Example 15 | 181 |

In FIGS. 10B to 10D, compared with other groups, Comparison 9 (administrated with Comparison 7 and radiotherapy at the same time) significantly decreased tumor volume and tumor weight by ˜70%. Interestingly, Example 15 (administrated with Example 13 of the vaccine of the present disclosure and radiotherapy at the same time) significantly decreased tumor volume and tumor weight by ˜90%. In FIGS. 10E and 10F and Tables 9 and 10, approximately 40% of colorectal cancer mice achieved a complete response (3/7) after treatment with Example 13 of the vaccine of the present disclosure, and the survival period was significantly extended. Moreover, there was no tumor growth in these tumor-free colorectal cancer mice after rechallenge with CT26 for 370 days, suggesting that vaccination with Example 13 of the vaccine of the present disclosure not only increased the therapeutic efficacy of radiotherapy but also inhibited tumor regrowth. The results in FIG. 10G show that the neoantigen-specific T-cell immune response was significantly increased in the solenocytes from Example 13 of the vaccine of the present disclosure vaccinated colorectal cancer mice. Therefore, the above results show that Example 13 of the vaccine of the present disclosure with the radiotherapy can achieve complete response and inhibit tumor recurrence.

### Examples 16-17

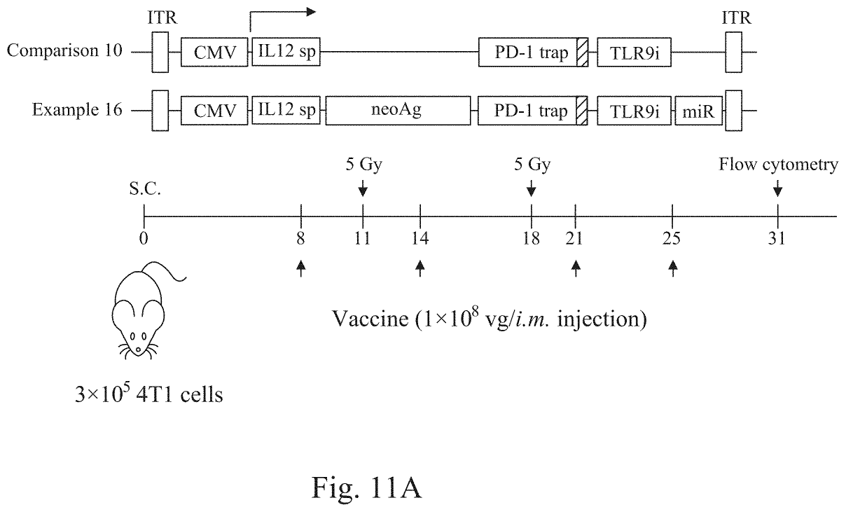

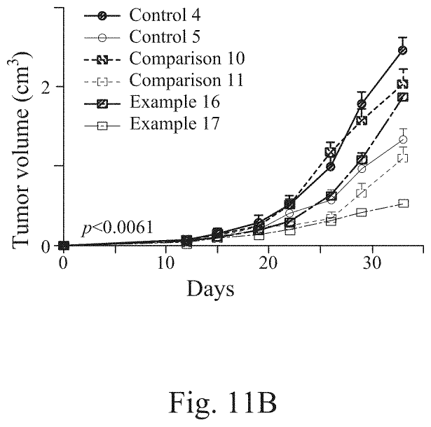

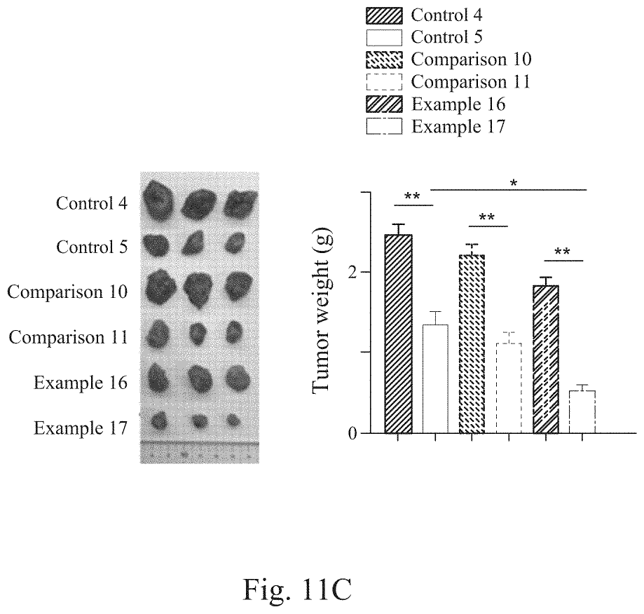

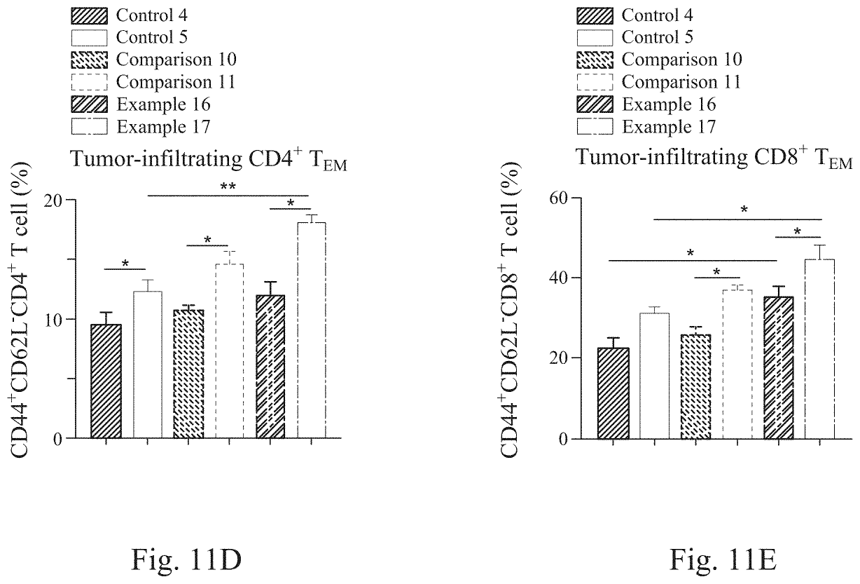

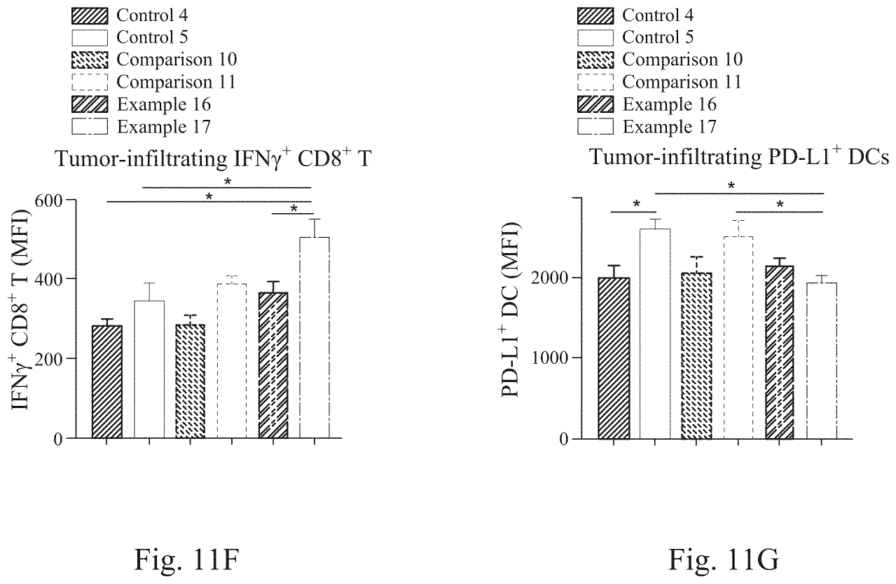

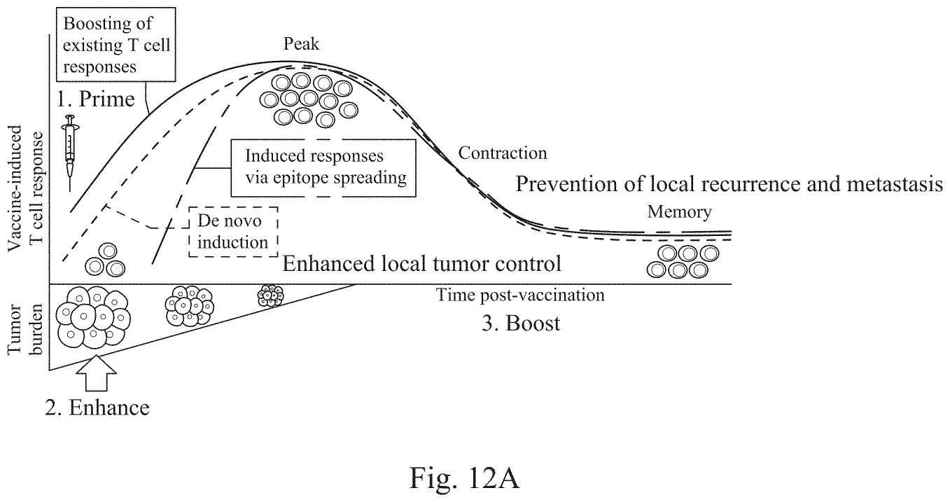

To further demonstrate that the vaccine of the present disclosure triggers high neoantigen immunogenicity to boost the therapeutic efficacy of radiotherapy, we generated still another vaccine carrying eight mutated TSAs, then vaccinated BALB/c mice bearing 4T1 tumors. Please refer to FIG. 11A and Table 11, FIG. 11A is a schematic view showing a construction and a treatment strategy of Example 16 of the vaccine of the present disclosure combined with the radiotherapy according to one example of the present disclosure in an animal treatment test, and Table 11 shows the treatment strategy of Examples 16-17, Comparisons 10-11 and Controls 4-5.

TABLE 11
The treatment strategy of Examples 16-
17, Comparisons 10-11 and Controls 4-5

| Group | Vaccine | Radiotherapy |
|---|---|---|
| Control 4 | PBS | — |
| Control 5 | PBS | 5 Gy × 2 |
| Comparison 10 | Comparison 10 | — |
| Comparison 11 | Comparison 10 | 5 Gy × 2 |
| Example 16 | Example 16 | — |
| Example 17 | Example 16 | 5 Gy × 2 |

In FIG. 11A, the peptides encoded by the transgene in Example 16 of the vaccine of the present disclosure includes the IL12 sp as the secretion signal peptide, a neoAg as the at least one tumor antigen, a PD-1 trap and a PD-L1 miRNA (presents as “miR” in FIG. 11A) as the least one co-inhibitory peptide, and the TLR9i as the TLR9 antagonist, and corresponding nucleotide fragments of the peptides are cloned into a CMV-driven pAAV-CMV expression vector. The amino acid sequence of the ID 2 sp is referenced as SEQ ID NO: 11. The neoAg includes the neoAgs 9-16 listed in Table 12. The amino acid sequence of the PD-1 trap is referenced as SEQ ID NO: 22, and the nucleic acid sequence of the PD-L1 miRNA is referenced as SEQ ID NO: 24. The TLR9i includes the amino acid sequences of SEQ ID NO: 20 and SEQ ID NO: 21. In addition, Comparison 10 is a pAAV-CMV vector including the nucleotide fragments encoding the IL12 sp, the PD-1 trap and the TLR9i but not including the nucleotide fragments encoding the tumor antigen and PD-L1 miRNA.

TABLE 12
The neoAgs in mouse mammary 4T1 cell line (hereinafter
referred to as “4T1 cell”)

| neoAg | Gene origin | neoAg sequence |
|---|---|---|
| 9 | Dhx58 | SEQ ID NO: 25 |
| 10 | Cand1 | SEQ ID NO: 26 |
| 11 | Wdr11 | SEQ ID NO: 27 |
| 12 | Pzp | SEQ ID NO: 28 |
| 13 | Gnpat | SEQ ID NO: 29 |
| 14 | Kbtbd2 | SEQ ID NO: 30 |
| 15 | Adamts9 | SEQ ID NO: 31 |
| 16 | Chsy1 | SEQ ID NO: 32 |

To demonstrate the therapeutic efficacy of the Example 16 of the vaccine of the present disclosure, a mammary cancer mouse model is established first. Six-week-old female BALB/c mice were inoculated subcutaneously 3×10⁵ 4 T1 cells with 20% matrigel (Corning, Union City, Calif., USA) to obtained BALB/c mice bearing 4T1 tumors, which are poorly immunogenic mammary cancer cells. After 8 days, the mammary cancer mice were randomly assigned into different groups, Example 16 of the vaccine of the present disclosure, Comparison 10 (1×10⁸ vg) and PBS were administered via intramuscular injection 4 times on Day 8, Day 14, Day 21 and Day 25, respectively. For radiotherapy, mammary cancer mice after complete anesthesia were placed in the irradiation field, the local tumors were received 5 Gy fractionated radiotherapy twice on Day 11 and Day 18. At the same time, the tumor volume was measured and calculated by the formula: V=(L×W²)/2 every 3 days, and flow cytometry was performed on Day 31.

Please refer to FIGS. 11B, 11C, 11D, 11E, 11F and 11G, which show the analysis results of the effect of Example 16 of the vaccine of the present disclosure in the treatment of mammary cancer, wherein * represents p<0.05, and ** represents p<0.01 using one-way ANOVA. In FIGS. 11B and 11C, Example 17 (administrated with Example 16 of the vaccine of the present disclosure and radiotherapy at the same time) decreases ˜80% tumor regression rate and tumor weight. In FIGS. 11D to 11F, the cell numbers of tumor-infiltrating CD4⁺, CD8⁺, CD4⁺T_{EM}, CD8⁺T_{EM} and IFNγ⁺CD8⁺ T cells were significantly increased within the residual tumors in Example 17. In FIG. 11G, the PD-L1 level in the tumor-infiltrating DCs was also significantly decreased in Example 17. Taken together, these results showed that Example 16 of the vaccine of the present disclosure can inhibit PD-L1 expression on DCs, leading to better antigen presentation and T-cell-mediated immune response. It indicates that Example 16 of the vaccine of the present disclosure increases the therapeutic efficacy of radiotherapy in a poorly immunogenic mammary animal model.

### Examples 18-22

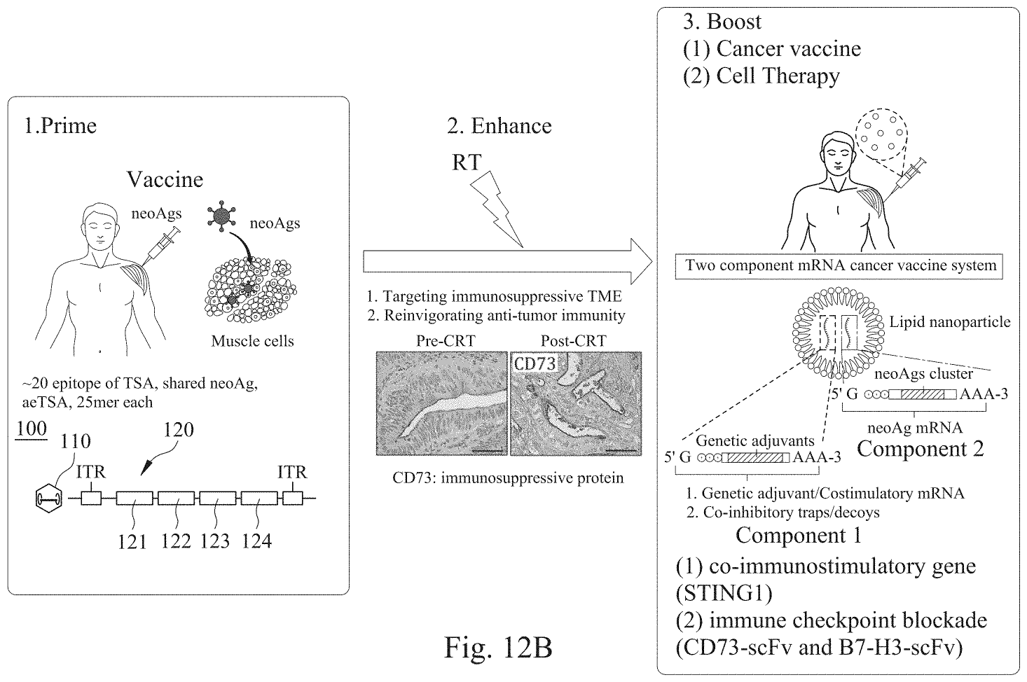

Please refer to FIGS. 12A and 12B. FIG. 12A is a schematic view showing a treatment strategy of a method for treating cancer by a cancer vaccine cocktail therapy according to one embodiment of the present disclosure. FIG. 12B is a schematic view showing a method for treating cancer by a cancer vaccine cocktail therapy according to one example of one embodiment of the present disclosure.

According to another embodiment, the method for treating cancer by a cancer vaccine cocktail therapy includes following steps. The vaccine according to the foregoing aspect is administered to a subject in need for a treatment of cancer to induce an immune priming against the at least one tumor antigen in the subject. An enhancer is administered to the subject to enhance local tumor control in the subject. A booster is administered to the subject to prevent local recurrence and metastasis in the subject.

According to the present disclosure, the at least one tumor antigen can be selected from a tumor-associated antigen (TAA), a tumor-specific antigen (TSA), an oncogenic mutation, an aberrantly expressed tumor-specific antigen (aeTSA) and a shared neoantigen (neoAg). The enhancer can be a radiation, a chemotherapeutic agent, an immunomodulating agent, a targeted therapy drug, an antibody drug, or a combination thereof. The booster can be a cancer vaccine including the at least one tumor antigen or a therapeutic cell including the at least one tumor antigen. Preferably, the cancer vaccine including the at least one tumor antigen can be a DC-based cancer vaccine or a virus-based cancer vaccine, and the therapeutic cell including the at least one tumor antigen can be a CIK (cytokine-induced killer cell), a DC-CIK or a neoAg-pulsed DC-CIK. The booster also can be a therapeutic cell including an immune checkpoint protein, an immunosuppressive factor and/or an immunostimulatory factor. Preferably, the therapeutic cell including the immune checkpoint protein can be a CAR-T cell, a CAR-NK cell or an adoptive T cell.

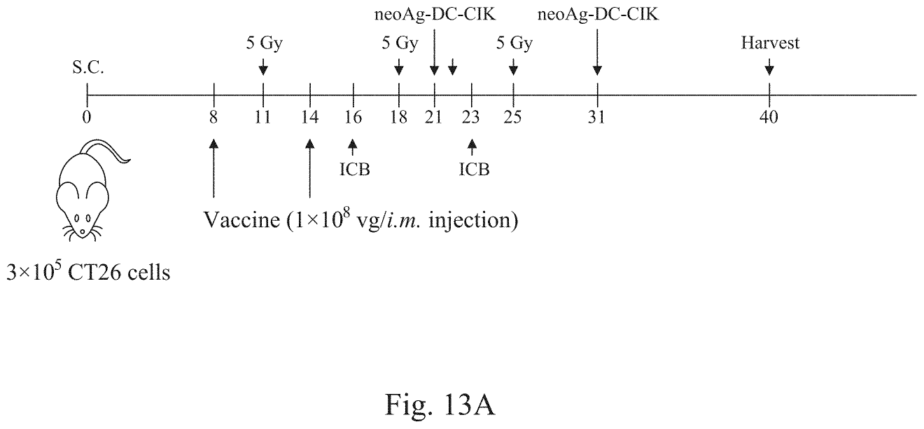

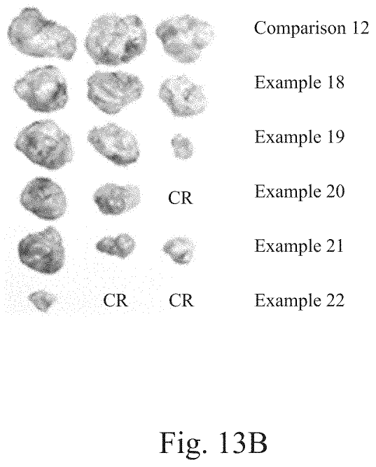

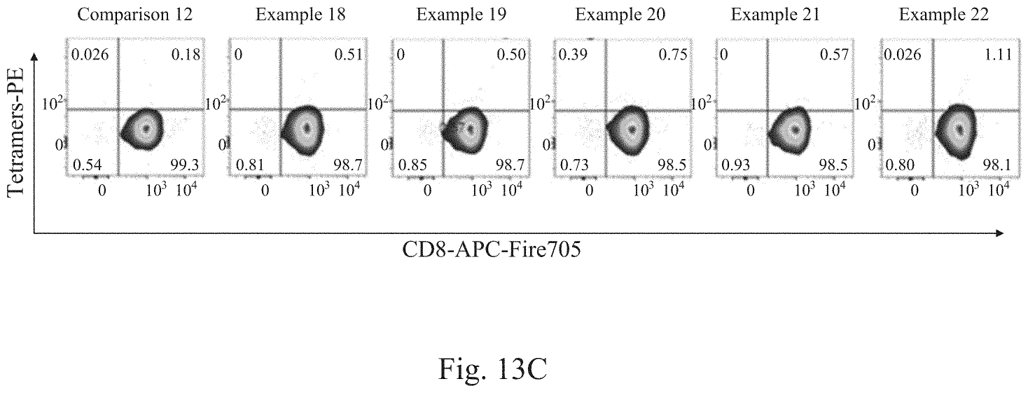

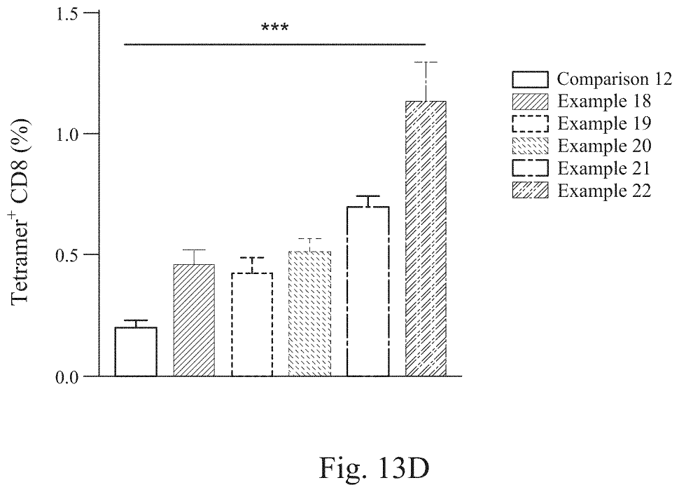

Please further refer to FIG. 13A and Table 13. FIG. 13A is a schematic view showing a treatment strategy of the cancer vaccine cocktail therapy according to one example of the present disclosure in an animal treatment test. Table 13 shows the treatment strategy of Examples 18-22 and Comparison 12.

TABLE 13
The treatment strategy of Examples 18-22 and Comparison 12

| Group | Vaccine | Enhancer | Booster | ICB |
|---|---|---|---|---|
| Comparison 12 | Comparison 7 | — | — | — |
| Example 18 | Example 13 | — | — | — |
| Example 19 | Example 13 | 5 Gy × 3 | — | — |
| Example 20 | Example 13 | 5 Gy × 3 | neoAg-DC-CIK | — |
| Example 21 | Example 13 | 5 Gy × 3 | — | αPD-1 |
| Example 22 | Example 13 | 5 Gy × 3 | neoAg-DC-CIK | αPD-1 |

To demonstrate the therapeutic efficacy of the cancer vaccine cocktail therapy of the vaccine of the present disclosure, colorectal cancer mice were randomly assigned into different groups. Example 13 of the vaccine of the present disclosure and Comparison 7 (1×10⁸ vg) were administered via intramuscular injection twice on Day 8 and Day 14, respectively. For radiotherapy as the enhancer, colorectal cancer mice after complete anesthesia were placed the right leg in the irradiation field, the local tumors were received 5 Gy fractionated radiotherapy 3 times on Day 11, Day 18 and Day 25. A neoAg-DC-CIK was used as the booster, and the neoAg-DC-CIK was administered via intramuscular injection twice on Day 21 and Day 31. In addition, αPD-1 as the immune checkpoint blockade (ICB) was administered twice on Day 16 and Day 23. At the same time, the tumor volume was measured and calculated by the formula: V=(L×W²)/2 every 3 days until Day 40 sacrificed.

Please refer to FIGS. 13B, 13C and 13D and Table 14. FIGS. 13B, 13C and 13D show the analysis results of the therapeutic effect of the cancer vaccine cocktail therapy of the present disclosure in the treatment of the colorectal cancer, wherein *** represents p<0.001 using one-way ANOVA. Table 14 shows the CR rate of Examples 18-22 and Comparison 12.

TABLE 14
The CR rate of Examples 18-22 and Comparison 12

| Group | CR rate |
|---|---|
| Comparison 12 | 0/6 |
| Example 18 | 0/6 |
| Example 19 | 0/6 |
| Example 20 | 2/6 |
| Example 21 | 0/6 |
| Example 22 | 5/6 |

In FIG. 13B, compared with other groups, Examples 19-22 significantly decreased tumor volume. Moreover, approximately 33% of colorectal cancer mice in Example 20 achieved a complete response (2/6), and approximately 83% of colorectal cancer mice in Example 22 achieved a complete response (5/6). The results in FIGS. 13C and 13D show that the tumor antigen-specific T-cell immune response was significantly increased in the solenocytes from colorectal cancer mice in Example 22. The results indicate that the cancer vaccine cocktail therapy of the present disclosure can achieve complete response and induce tumor antigen-specific T cell immune responses.

In summary, the vaccine of the present disclosure coexpresses the at least one co-inhibitory peptide and TLR9 antagonist to increase the at least one tumor antigen expression to activate tumor antigen specific T-cell responses. Therefore, the vaccine of the present disclosure holds great promise and advantages due to its clinical safety and low immunological clearance prior to sufficient transgene expression. In addition, the vaccine of the present disclosure can increase the therapeutic efficacy of radiotherapy against cancer. Thus, radiotherapy synergistically increases the therapeutic efficacy of the vaccine of the present disclosure including the co-inhibitory peptide-armed tumor antigen, providing a novel, safe and efficient tumor antigen-based immunotherapy. Furthermore, the cancer vaccine cocktail therapy of the present disclosure including administering the vaccine of the present disclosure, the enhancer and the booster can effectively inhibit tumor growth and inhibit tumor recurrence.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A vaccine, comprising:
a vector; and
a transgene encoding a plurality of peptides and packaged in the vector, wherein the peptides in order comprise:
a secretion signal peptide;
at least one tumor antigen, wherein the at least one tumor antigen is a subtraction of tumor and normal cell antigens;
at least one co-inhibitory peptide, wherein the at least one co-inhibitory peptide comprises programmed death-ligand 1 (PD-L1) antagonist, programmed cell death protein-1 (PD-1) antagonist or a cytotoxic T-lymphocyte-associated protein 4 (CTLA4) antagonist; and
a toll-like receptor 9 (TLR9) antagonist.

2. The vaccine of claim 1, further comprising a co-stimulatory peptide between the at least one co-inhibitory peptide and the TLR9 antagonist, wherein the co-stimulatory peptide is selected from a granulocyte-macrophage colony-stimulating factor (GM-CSF), interleukin 12 (IL12) and interferon (IFNs).

3. The vaccine of claim 1, wherein the vector is a vaccinia viral vector, an adeno-associated virus (AAV) vector or a nanoparticle.

4. The vaccine of claim 1, wherein the secretion signal peptide is an interleukin 2 signal peptide (IL2 sp) or an interleukin 12 signal peptide (IL12 sp).

5. The vaccine of claim 1, wherein the at least one tumor antigen is selected from a tumor-associated antigen (TAA), a tumor-specific antigen (TSA), an oncogenic mutation, an aberrantly expressed tumor-specific antigen (aeTSA) and a shared neoantigen (neoAg).

6. The vaccine of claim 1, wherein the at least one tumor antigen is selected by comparing whole exome sequencing of matched tumor and normal cell DNA from the patient to identify tumor-specific somatic mutations.

7. The vaccine of claim 1, wherein PD-L1 antagonist comprises a PL-L1 trap and a PD-1 peptide.

8. The vaccine of claim 1, wherein the PD-1 antagonist comprises a PD-1 trap and a PD-L1/PD-L2 peptide.

9. The vaccine of claim 1, wherein the CTLA4 antagonist comprises a CTLA4 trap and an antagonistic antibody against CTLA4.

10. The vaccine of claim 1, wherein the TLR9 antagonist is selected from a CpG oligonucleotide TLR9 binding domain, a TLR decoy peptide and a CpG binding sequence.

11. A method for treating cancer comprising administering the vaccine of claim 1 to a subject in need for a treatment of cancer to induce an anti-tumor immune response in the subject.

12. The method of claim 11, further administering radiotherapy to the subject.

13. A method for treating cancer by a cancer vaccine cocktail therapy comprising:
administering the vaccine of claim 1 to a subject in need for a treatment of cancer to induce an immune priming against the at least one tumor antigen in the subject;
administering an enhancer to the subject to enhance local tumor control in the subject; and
administering a booster to the subject to prevent local recurrence and metastasis in the subject.

14. The method of claim 13, wherein the at least one tumor antigen is selected from a tumor-associated antigen (TAA), a tumor-specific antigen (TSA), an oncogenic mutation, an aberrantly expressed tumor-specific antigen (aeTSA) and a shared neoantigen (neoAg).

15. The method of claim 13, wherein the enhancer is a radiation, a chemotherapeutic agent, an immunomodulating agent, a targeted therapy drug, an antibody drug, or a combination thereof.

16. The method of claim 13, wherein the booster is a cancer vaccine comprising the at least one tumor antigen or a therapeutic cell comprising the at least one tumor antigen.

17. The method of claim 16, wherein the cancer vaccine is a DC-based cancer vaccine or a virus-based cancer vaccine, and the therapeutic cell is a CIK (cytokine-induced killer cell), a DC-CIK or a neoAg-pulsed DC-CIK.

18. The method of claim 16, wherein the at least one tumor antigen is selected from a tumor-associated antigen (TAA), a tumor-specific antigen (TSA), an oncogenic mutation, an aberrantly expressed tumor-specific antigen (aeTSA) and a shared neoantigen (neoAg).

19. The method of claim 13, wherein the booster is a therapeutic cell comprising an immune checkpoint protein, an immunosuppressive factor and/or an immunostimulatory factor.

20. The method of claim 19, wherein the therapeutic cell is a CAR-T cell, a CAR-NK cell or an adoptive T cell.
